SEQUENCE LISTING (7 sequences)

SEQ ID NO: 1 - DNA, 300 bp, Enterotoxigenic Escherichia coli
  source 1..300: mol_type = genomic DNA
ggagcatctcagttcttcaaagacaattgtaatcgtaccaccgcttctctggtcgagggtgtagagctgaccaaatatatcagcgacattaataacaacaccgacgggatgtatgtagtgagttccacaggcggagtatggcgtatcagtcgtgccaaagactatccggataacgtcatgacagccgaaatgcgcaaaattgctatggcggcagttctgtctggtatgcgcgtgaacatgtgtgctagtccagcaagttcgcctaacgtgatctgggccatcgaactggaagcagaataa

SEQ ID NO: 2 - protein, 8 aa, synthetic construct
GGSGGGSG

SEQ ID NO: 3 - protein, 38 aa, Autographa californica nucleopolyhedrovirus
MLLVNQSHQGFNKEHTSKMVSAIVLYVLLAAAAHSAFA

SEQ ID NO: 4 - DNA, 114 bp, Autographa californica nucleopolyhedrovirus
  source 1..114: mol_type = genomic DNA
atgctgctcgtcaaccagtcacatcagggattcaacaaagaacatacctcaaaaatggtgtcggctatcgtcctctacgtcctcctcgctgccgctgcccactctgctttcgct

SEQ ID NO: 5 - DNA, 27 bp, synthetic construct
  source 1..27: mol_type = other DNA
caccatcatcaccaccaccaccaccac

SEQ ID NO: 6 - DNA, 576 bp, severe acute respiratory syndrome coronavirus 2
  source 1..576: mol_type = genomic DNA
cccaacattactaacctctgcccattcggagaagttttcaacgccacaagattcgcctctgtctacgcctggaaccgcaaacgcattagcaactgcgtcgccgactactctgtgctgtacaactctgcttcattctccaccttcaagtgctacggcgtgagccctaccaagctgaacgacctgtgcttcactaacgtctacgccgactctttcgtgatcaggggagacgaggtcagacagatcgccccaggacagaccggcaagatcgctgactacaactacaagctgcctgacgacttcactggctgcgtgatcgcttggaacagcaacaacctggactctaaggtcggtggcaactacaactacctgtacaggctgttcagaaagtccaacctgaagcccttcgagagggacatctcaaccgaaatctaccaggccggttccaccccatgcaacggcgtggaaggattcaactgctacttccctctgcaatcatacggtttccagcccaccaacggtgtcggctaccagccataccgcgtggtcgtgctgtccttcgagctgctgcacgccccc

SEQ ID NO: 7 - DNA, 729 bp, Enterotoxigenic Escherichia coli
  source 1..729: mol_type = genomic DNA
aacgactacttccgcgctgacagccgtactcctgacgaagtgcgccgttctggcggactgatcccccgcggacaggacgaggcctacgaacgtggcaccccaatcaacattaacctgtacgaccacgccaggggaaccgctactggtaacactagatacaacgacggttacgtcagcaccactaccactctgcgccaggctcacttcctgggccagaacatgctgggtggctacaacgagtactacatctacgtcgtggctgccgctccaaacctgttcgacgtcaacggtgtgctgggcaggtactcaccttacccctccgagaacgaatacgccgctctgggaggtatccctctgtcacagatcatcggttggtacagagtgtccttcggcgctatcgaaggcggaatgcaccgcaaccgtgactaccgtcgcgacctgttcaggggcctgagcgccgctcctaacgaggacggataccgtatcgccggcttcccagacggattccctgcttgggaggaagtcccctggagggagttcgctcccaactcttgcctgccaaacaacaaggcctccagcgacaccacttgcgcttcactgaccaacaagctgtcccagcacgacctggctgacttcaagaagtacatcaagcgtaagttcactctgatgactctgctctcaatcaacaacgatggtttcttctccaacaacggcggcaaggatgaactg

(19) United States
(12) Patent Application Publication
(10) Pub. No.: US 2023/0149533 A1
(43) Pub. Date: May 18, 2023

(54) SARS-COV-2 MUCOSAL VACCINE COMPOSITION, PREPARATION AND USE THEREOF

(71) Applicant: National Tsing Hua University, Hsinchu (TW)

(72) Inventors: SUH CHIN WU, Hsinchu (TW); PIN-HAN CHOU, Hsinchu (TW); HE-CHIN HSIEH, Hsinchu (TW)

(21) Appl. No.: 17/881,892

(22) Filed: Aug. 5, 2022

(30) Foreign Application Priority Data
Nov. 18, 2021 (TW) 110143063

(51) Int. Cl. A61K 39/215; A61P 31/14

(52) U.S. Cl. CPC A61K 39/215; A61P 31/14; A61K 2039/6037

(57) ABSTRACT

The invention provides a SARS-CoV-2 mucosal vaccine composition, preparation, and use thereof. The SARS-CoV-2 mucosal vaccine composition comprises an antigen fusion protein which includes a SARS-CoV-2 antigen and a Type IIb heat-labile enterotoxin A subunit from Escherichia coli. Immunization with the antigen fusion protein elicits cellular and humoral immune responses, including systemic and mucosal immune responses, against SARS-CoV-2 in a subject, and thus protects the subject from viral infection.

### CROSS-REFERENCE TO RELATED APPLICATION

This application claims priority of Taiwan Patent Application No. 110143063, filed on Nov. 18, 2021, the content of which is incorporated herein in its entirety by reference.

### STATEMENT REGARDING SEQUENCE LISTING

[0001a] The sequence listing associated with this application is provided in text format in lieu of a paper copy and is hereby incorporated by reference into the specification. The name of the XML file containing the sequence listing is 110F0400-IE_Sequence_listing. The XML file is 8567 bytes; was created on Aug. 5, 2022.

### BACKGROUND OF THE INVENTION

### Field of the Invention

The present invention relates to a vaccine composition, preparation, and use thereof; in particular, the present invention relates to a severe acute respiratory syndrome coronavirus 2 (SARS-CoV-2) mucosal vaccine composition, preparation, and use thereof using an antigen fusion protein.

### The Prior Art

Severe acute respiratory syndrome coronavirus 2 (SARS-CoV-2) is an enveloped positive-sense single-stranded RNA virus, belonging to severe acute respiratory syndrome-related coronavirus of Betacolonavirus of the Coronaviradae family. The virus particles are round or elliptical, with a diameter of about 80-120 nanometers. The virus particles are coated by a double layer of phospholipids provided by the host cell and mainly contain four structural proteins including envelope proteins (E protein), membrane protein (M protein), nucleocapsid, and spike protein (S protein). SARS-CoV-2 caused Coronavirus disease 2019 (COVID-19) that broke out at the end of 2019. SARS-CoV-2 can invade the human body through the upper respiratory tract by infecting a variety of cells expressing ACE2 on the surface; and the main infected organs include lungs, heart, kidney, and other major organs.

To avoid the huge health and economic losses due to SARS-CoV-2, medical researchers have focused on the development of SARS-CoV-2 vaccines. The currently commonly used vaccines include mRNA vaccine, deactivated virus vaccines, attenuated vaccines, viral vector vaccines, and protein subunit vaccines. Protein subunit vaccines are part of viral proteins which are expressed using other biological platforms. Protein subunit vaccines are safer than virus vector vaccines, deactivated virus vaccines, or attenuated vaccines, but the immunogenicity of protein subunits is usually low, so protein subunits are difficult to elicit adequate immune responses and need additional adjuvants to enhance the immune system of individuals to provide sufficient protection when being used as vaccines.

According to the administration routes, vaccines can be classified into intramuscular vaccines and mucosal vaccines, etc. Mucosal vaccines may be administered via nasal, sublingual, oral, rectal, and vaginal routes. Mucosal vaccines induce both mucosal and systemic immune responses against antigens, wherein inducing mucosal immune responses of the respiratory tract can provide individuals with first-line protection against foreign pathogens. However, the mucosal system contains certain immune tolerance to inhibit the overreaction of the immune system. Therefore, in order to trigger sufficient mucosal immunity, the antigens need to be combined with adjuvants to break through the tolerance. Common mucosal vaccine adjuvants include cholera toxin (CT), heat-labile enterotoxin (LT), unmethylated CpG dinucleotides, monophosphoryl lipid A (MPL), and Toll-like receptor stimulants.

Heat-labile enterotoxins are bacterial protein toxins, which are classified into the Type I and Type II subfamilies according to the amino acid sequence and specific binding to gangliosides. The Type II subfamily is further divided into three subgroups, including Type IIa, Type IIb, and Type IIc. The heat-labile enterotoxin consists of A and B subunits. The A subunit has the adenosine diphosphate (ADP)-ribosylating activity and the B-subunit pentamer can bind to glycoproteins on the surface of eukaryotic cells to enable the cell entry of enterotoxins. Because the action of the LT A subunit in cells reduces intestinal water absorption and leads to diarrhea, the LT-based vaccine adjuvants used in previous studies are usually the LT holotoxins containing a mutant A subunit or the pentamer of LT B subunit. There is a lack of knowledge regarding preparing an effective SARS-CoV-2 mucosal vaccine by a more simplified method, for example, constructing a single fusion protein by conjugating the wild-type LT A subunit with an influenza virus antigen.

### SUMMARY OF THE INVENTION

To solve the foregoing problem, one objective of the present invention is to provide a severe acute respiratory syndrome coronavirus 2 (SARS-CoV-2) mucosal vaccine composition, comprising an antigen fusion protein, wherein the antigen fusion protein comprises a SARS-CoV-2 antigen and a Type IIb heat-labile enterotoxin A subunit from an Escherichia coli.

Another objective of the present invention is to provide a method of preparing a SARS-CoV-2 mucosal vaccine composition, comprising the steps of: (a) preparing an antigen fusion protein which comprises a SARS-CoV-2 antigen and a Type IIb heat-labile enterotoxin A subunit from an Escherichia coli; and (b) mixing the antigen fusion protein with a pharmaceutically acceptable carrier to obtain a SARS-CoV-2 mucosal vaccine composition.

The other objective of the present invention is to provide a method of treating or preventing SARS-CoV-2 infection, comprising administering to a subject in need thereof a SARS-CoV-2 mucosal vaccine composition comprising an effective amount of an antigen fusion protein which comprises a SARS-CoV-2 antigen and a Type IIb heat-labile enterotoxin A subunit (RBD-LTIIbA) from an Escherichia coli.

In one embodiment of the present invention, the SARS-CoV-2 antigen is a spike protein.

In one embodiment of the present invention, the SARS-CoV-2 antigen is a

receptor-binding domain (RBD) of the spike protein.

In one embodiment of the present invention, an N-terminal region of the SARS-CoV-2 antigen further comprises a poly-histidine segment and/or a signal peptide segment of an envelope glycoprotein.

In one embodiment of the present invention, a binding stability between the RBD and an angiotensin-converting enzyme 2 (ACE2) is not affected by the structure of the antigen fusion protein.

In one embodiment of the present invention, the SARS-CoV-2 mucosal vaccine composition comprises at least 45 µg of the antigen fusion protein.

In one embodiment of the present invention, the antigen fusion protein elicits high titer antigen-specific antibodies and/or neutralizing antibodies against SARS-CoV-2; and the antigen-specific antibodies and the neutralizing antibodies are IgG antibodies and/or IgA antibodies.

In one embodiment of the present invention, the antigen fusion protein elicits a T-cell-related immune response.

In one embodiment of the present invention, the T-cell-related immune response includes secretions of interferon-y (IFN-y), interleukin-5 (IL-5), and interleukin-17A (IL-17A), or any combinations thereof.

In one embodiment of the present invention, the SARS-CoV-2 mucosal vaccine composition is administered through a nasal cavity of the subject in need thereof.

In the SARS-CoV-2 mucosal vaccine composition of the present invention, a protein subunit of the RBD-LTIIbA fusion protein is used as an antigen to form a vaccine composition with a built-in adjuvant. In the case of fusion with LTIIbA, the structure of the RBD-LTIIbA fusion protein of the present invention will not affect the binding stability between the RBD and ACE2. When the SARS-CoV-2 mucosal vaccine composition of the present invention is applied to a subject, such as nasal injection, the SARS-CoV-2 mucosal vaccine composition can effectively elicit the humoral and cellular immune responses against SARS-CoV-2 in the subject without any additional adjuvants, including eliciting antigen-specific and the neutralizing IgG antibodies and IgA antibodies in blood and broncho-alveolar mucosa against SARS-CoV-2, and increasing cytokines secreted by T cells such as IFN-y, IL-5, and IL-17A, to effectively enhance the ability of the subject to prevent the attack/infection of SARS-CoV-2. The SARS-CoV-2 mucosal vaccine composition of the present invention can effectively elicit systemic immunity and mucosal immunity as well, and especially stimulate mucosal immune responses of the respiratory tract, so as to provide the first-line protection against foreign pathogens directly. Therefore, the SARS-CoV-2 mucosal vaccine composition of the present invention can provide subjects with effective protection against SARS-CoV-2 infection.

The embodiments of the present invention are further described with the following drawings. The following embodiments are given to illustrate the present invention and are not intended to limit the scope of the present invention, and one with ordinary skill in the art can make some modifications and refinements without departing from the spirit and scope of the present invention. Therefore, the scope of the present invention is defined by the scope of the appended claims.

### BRIEF DESCRIPTION OF THE DRAWINGS

FIG. 1A shows a schematic view of the DNA construct of the RBD-LTIIbA fusion protein of the present invention; wherein, GP67 represents the nucleic acid fragment corresponding to the signal peptide segment of the envelope glycoprotein; 9*His represents the nucleic acid fragment corresponding to the histidine tag consisted of nine consecutive histidines; RBD represents the nucleic acid fragment corresponding to the receptor-binding domain of the spike protein of SARS-CoV-2; GS-linker represents the nucleic acid fragment corresponding to the short peptide segment of the GS linker molecule; LTIIb A represents the gene of the Type IIb heat-labile enterotoxin A subunit from Escherichia coli.

FIG. 1B shows a schematic view of the DNA construct of the recombinant RBD protein; wherein, GP67 represents the nucleic acid fragment corresponding to the signal peptide segment of the envelope glycoprotein; 9*His represents the nucleic acid fragment corresponding to the histidine tag consisted of nine consecutive histidines; RBD represents the nucleic acid fragment corresponding to the receptor-binding domain of the spike protein of SARS-CoV-2.

FIG. 1C shows the analyzing results of the recombinant RBD protein of FIG. 1B and the RBD-LTIIbA fusion protein of FIG. 1A by staining the gel of SDS-PAGE (left) and by Western blotting assay (right); wherein, RBD represents the recombinant RBD protein; RBD-LTIIbA represents the RBD-LTIIbA fusion protein of the present invention.

FIG. 1D shows the binding affinity of the recombinant RBD protein or the RBD-LTIIbA fusion protein of the present invention to the ACE2 protein determined by enzyme-linked immunosorbent assay (ELISA); wherein, RBD represents the recombinant RBD protein; RBD-LTIIbA represents the RBD-LTIIbA fusion protein of the present invention; B represents the binding substrate (reads of absorbance); F represents the concentration of free receptor; Bmax represents the maximum signal intensity of the binding substrate (represented by the signal intensity at OD₄₅₀, i.e. the signal from ACE2); Kd represents the dissociation constant.

FIG. 2A shows the titers of anti-RBD IgG antibodies in the serum of mice after being intranasally immunized with the 1^{st} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of polyinosinic acid-polycytidylic acid (poly(I:C)); Ad-S represents the serum from the positive control group of mice immunized with 10⁸ PFU of the adenovirus vector.

FIG. 2B shows the titers of anti-RBD IgA antibodies in the serum of mice after being intranasally immunized with the 1^{st} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 2C shows the titers of anti-RBD IgG antibodies in the serum of mice after being intranasally immunized with the 2^{nd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 2D shows the titers of anti-RBD IgA antibodies in the serum of mice after being intranasally immunized with the 2^{nd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 2E shows the titers of anti-RBD IgG antibodies in the serum of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 2F shows the titers of anti-RBD IgA antibodies in the serum of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 2G shows the titers of anti-RBD IgG antibodies in the broncho-alveolar lavage fluids (BALFs) of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 2H shows the titers of anti-RBD IgA antibodies in the BALFs of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 2I shows the titers of total IgA antibodies in the BALFs of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 3A shows the results of the serum of mice after being intranasally immunized with the 3^{rd} dose of the mucosal vaccine composition of the present invention against the original Wuhan-Hu-1 strain of SARS-CoV-2 by pseudovirus-based neutralization assay; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 3B shows the results of the serum of mice after being intranasally immunized with the 3^{rd} dose of the mucosal vaccine composition of the present invention against the Alpha mutant strain of SARS-CoV-2 (B.1.1.7) by pseudovirus-based neutralization assay; wherein, PBS represents the serum from the negative control group of mice immunized with PBS solution only; RBD-LTIIbA represents the serum from the experimental group of mice immunized with 45 µg of the RBD-LTIIbA fusion protein of the present invention; Ad-S represents the serum from the positive control group of mice immunized with 10⁸ PFU of the adenovirus vector.

FIG. 3C shows the results of the serum of mice after being intranasally immunized with the 3^{rd} dose of the mucosal vaccine composition of the present invention against the Beta mutant strain of SARS-CoV-2 (B.1.351) by pseudovirus-based neutralization assay; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 3D shows the results of the serum of mice after being intranasally immunized with the 3^{rd} dose of the mucosal vaccine composition of the present invention against the Delta mutant strain of SARS-CoV-2 (B.1.617.2) by pseudovirus-based neutralization assay; wherein, PBS represents the serum from the negative control group of mice immunized with PBS solution only; RBD-LTIIbA represents the serum from the experimental group of mice immunized with 45 µg of the RBD-LTIIbA fusion protein of the present invention; Ad-S represents the serum from the positive control group of mice immunized with 10⁸ PFU of the adenovirus vector.

FIG. 3E shows the IC50 of the neutralizing effects of the serum of mice after being intranasally immunized with the 3^{rd} dose of the mucosal vaccine composition of the present invention against the original Wuhan-Hu-1 strain, the Alpha mutant strain, the Beta mutant strain, and the Delta mutant strain of SARS-CoV-2 by pseudovirus-based neutralization assay; wherein, PBS represents the serum from the negative control group of mice immunized with PBS solution only; RBD-LTIIbA represents the serum from the experimental group of mice immunized with 45 µg of the RBD-LTIIbA fusion protein of the present invention; Ad-S represents the serum from the positive control group of mice immunized with 10⁸ PFU of the adenovirus vector.

FIG. 4A shows the concentration of IFN-y in splenocytes (SPLs) of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 4B shows the concentration of IFN-y in cervical lymph nodes (CLNs) of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 4C shows the concentration of IL-5 in SPLs of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 4D shows the concentration of IL-5 in CLNs of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 4E shows the concentration of IL-17A in SPLs of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 4F shows the concentration of IL-17A in CLNs of mice after being intranasally immunized with the 3^{rd} dose of the mucosal vaccine composition of the present invention by ELISA; wherein, PBS represents the serum from the negative control group of mice immunized with PBS solution only; RBD represents the serum from the comparison group of mice immunized with 20 µg of the recombinant RBD protein; RBD+LTIIbB represents the serum from the comparison group of mice immunized with 20 µg of the recombinant RBD protein and 5 µg of the LTIIb-B5 protein; RBD-LTIIbA represents the serum from the experimental group of mice immunized with 45 µg of the RBD-LTIIbA fusion protein of the present invention; RBD+poly(I:C) represents the serum from the comparison group of mice immunized with 20 µg of the recombinant RBD protein and 2 µg of poly(I:C); Ad-S represents the serum from the positive control group of mice immunized with 10⁸ PFU of the adenovirus vector.

FIG. 5A shows the titers of anti-RBD IgG antibodies in the serum of hamsters after being intranasally immunized with the 1^{st} dose of the mucosal vaccine composition of the present invention; wherein, PBS represents the serum from the negative control group of hamsters immunized with PBS solution only; RBD-LTIIbA represents the serum from the experimental group of hamsters immunized with 45 µg (low-dose) or 90 µg (high-dose) of the RBD-LTIIbA fusion protein of the present invention.

FIG. 5B shows the titers of anti-RBD IgG antibodies in the serum of hamsters after being intranasally immunized with the 2^{nd} dose of the mucosal vaccine composition of the present invention; wherein, PBS represents the serum from the negative control group of hamsters immunized with PBS solution only; RBD-LTIIbA represents the serum from the experimental group of hamsters immunized with 45 µg (low-dose) or 90 µg (high-dose) of the RBD-LTIIbA fusion protein of the present invention.

FIG. 5C shows the titers of anti-RBD IgG antibodies in the serum of hamsters after being intranasally immunized with the 3^{rd} dose of the mucosal vaccine composition of the present invention; wherein, PBS represents the serum from the negative control group of hamsters immunized with PBS solution only; RBD-LTIIbA represents the serum from the experimental group of hamsters immunized with 45 µg (low-dose) or 90 µg (high-dose) of the RBD-LTIIbA fusion protein of the present invention.

FIG. 5D shows the results of the serum of hamsters after being intranasally immunized with the 2^{nd} dose of the mucosal vaccine composition of the present invention against SARS-CoV-2 by pseudovirus-based neutralization assay; wherein, PBS represents the serum from the negative control group of hamsters immunized with PBS solution only; RBD-LTIIbA represents the serum from the experimental group of hamsters immunized with 45 µg (low-dose) or 90 µg (high-dose) of the RBD-LTIIbA fusion protein of the present invention.

FIG. 5E shows the results of the serum of hamsters after being intranasally immunized with the 3^{rd} dose of the mucosal vaccine composition of the present invention against SARS-CoV-2 by pseudovirus-based neutralization assay; wherein, PBS represents the serum from the negative control group of hamsters immunized with PBS solution only; RBD-LTIIbA represents the serum from the experimental group of hamsters immunized with 45 µg (low-dose) or 90 µg (high-dose) of the RBD-LTIIbA fusion protein of the present invention.

FIG. 5F shows the IC50 of the neutralizing effects of the serum of hamsters after being intranasally immunized with the mucosal vaccine composition of the present invention against SARS-CoV-2 by pseudovirus-based neutralization assay; wherein, PBS represents the serum from the negative control group of hamsters immunized with PBS solution only; RBD-LTIIbA represents the serum from the experimental group of hamsters immunized with 45 µg (low-dose) or 90 µg (high-dose) of the RBD-LTIIbA fusion protein of the present invention.

FIG. 6A shows the results of the body weight change of hamsters after the virus challenge. Hamsters were intranasally immunized with three doses of the mucosal vaccine composition of the present invention and then were challenged with SARS-CoV-2 (hCoV-19/Taiwan/4/2020). The body weight of hamsters was tracked for 3 days; wherein, PBS represents the serum from the negative control group of hamsters immunized with PBS solution only; RBD-LTIIbA represents the serum from the experimental group of hamsters immunized with 45 µg (low-dose) or 90 µg (high-dose) of the RBD-LTIIbA fusion protein of the present invention; dpi represents day(s) post-infection.

FIG. 6B shows the results of the body weight change of hamsters after the virus challenge. Hamsters were intranasally immunized with three doses of the mucosal vaccine composition of the present invention and then were challenged with SARS-CoV-2 (hCoV-19/Taiwan/4/2020). The body weight of hamsters was tracked for 6 days; wherein, PBS represents the serum from the negative control group of hamster immunized with PBS solution only; RBD-LTIIbA represents the serum from the experimental group of hamster immunized with 45 µg (low-dose) or 90 µg (high-dose) of the RBD-LTIIbA fusion protein of the present invention; dpi represents day(s) post-infection.

FIG. 6C shows the results of fifty-percent tissue culture infectious dose (TCID50) in lung tissues of hamsters, which were intranasally immunized with the 43^{rd} dose of the mucosal vaccine composition of the present invention and then were challenged with SARS-CoV-2 (hCoV-19/Taiwan/4/2020); wherein, PBS represents the serum from the negative control group of hamster immunized with PBS solution only; RBD-LTIIbA represents the serum from the experimental group of hamster immunized with 45 µg (low-dose) or 90 µg (high-dose) of the RBD-LTIIbA fusion protein of the present invention; dpi represents day(s) post-infection.

FIG. 6D shows the results of TCID50 in nasal wash of hamsters, which were intranasally immunized with three doses of the mucosal vaccine composition of the present invention and then were challenged with SARS-CoV-2 (hCoV-19/Taiwan/4/2020); wherein, PBS represents the serum from the negative control group of hamster immunized with PBS solution only; RBD-LTIIbA represents the serum from the experimental group of hamster immunized with 45 µg (low-dose) or 90 µg (high-dose) of the RBD-LTIIbA fusion protein of the present invention; dpi represents day(s) post-infection.

FIG. 6E shows the sectional stained images of lung tissues of hamsters, which were intranasally immunized with the 3^{rd} dose of the mucosal vaccine composition of the present invention and then were challenged with SARS-CoV-2 (hCoV-19/Taiwan/4/2020); wherein, PBS represents the serum from the negative control group of hamster immunized with PBS solution only; RBD-LTIIbA represents the serum from the experimental group of hamster immunized with 45 µg (low-dose) or 90 µg (high-dose) of the RBD-LTIIbA fusion protein of the present invention; dpi represents day(s) post-infection.

### DETAILED DESCRIPTION OF THE PREFERRED EMBODIMENT

All experimental results were analyzed by GraphPad Prism v6.01. The titers of anti-RBD IgG antibodies and anti-RBD IgA antibodies were analyzed by Dunn’s multiple comparisons test. The concentration of IFN-y, the concentration of IL-5, and the concentration of IL-17A were analyzed by Tukey’s test. Statistical significance in all experimental results was expressed as follows: *p < 0.05; **p < 0.01; and ***p < 0.001. All experiments were performed at least twice.

### Definition

Herein, the data provided represent experimental values that can vary within a range of ±20%, preferably within ±10%, and most preferably within ±5%.

According to the present invention, the operating procedures and parameter conditions of gene cloning are within the professional literacy and routine techniques of one with ordinary skill in the art.

Herein, the term “RBD-LTIIbA” is equivalent to the term “RBD-LTIIbA fusion protein” unless otherwise specified.

### Material and Method

### Preparation of the Recombinant Protein of Type IIb Heat-Labile Enterotoxin B Subunit (LTIIb-B5) of Escherichia Coli

For expression of the pentameric recombinant LTIIb-B5 protein, LTIIb-B5 gene (SEQ ID NO: 1) from enterotoxigenic Escherichia coli (ETEC) was codon-optimized and cloned into a pET22b(+) vector to construct an LTIIb-BS-pET22b(+) plasmid. Next, E. coli BL21 cells (DE3) (Invitrogen) were transformed with the LTIIb-BS-pET22b(+) plasmid and cultured overnight at 37° C. in Luria-Bertani (LB) medium containing ampicillin. The overnight culture was inoculated into ampicillin-containing LB medium and incubated at 37° C. until an absorbance of 0.4-0.6 at 600 nm (OD 600) was reached, and an additional 4-hour incubation was carried out at 37° C. after isopropyl βP-D-1-thiogalactopyranoside (IPTG) was added to induce the expression of recombinant LTIIb-B5 protein. The cell pellet was collected by centrifugation (10,000 rpm, 10 minutes, 4° C.).

For protein purification, the abovementioned cell pellet was resuspended in buffer A (300 mM Tris, 50 mM sodium chloride, 10 mM imidazole, 5% glycerol, pH 7.2) containing phenylmethane-sulfonyl fluoride (PMSF) and lysed at high pressure (15 Kpsi). The cell lysate was centrifuged at 10,000 rpm for 10 minutes at 4° C., and the pellet was collected and mixed with buffer A containing 8 M urea. The mixture was centrifuged at 10,000 rpm for 10 minutes at 4° C., and the supernatant was collected and mixed overnight with nickel-chelating resin (TOSOH). A column was packed with the resin mixture, washed with buffer A containing 0.5% Triton X-100, and eluted with 30-40% buffer B (300 mM Tris, 50 mM sodium chloride, 500 mM imidazole, 5% glycerol, pH 7.2-7.5) to obtain the recombinant LTIIb-B5 protein. The fractions of purified recombinant LTIIb-B5 protein were transferred to a dialysis bag with a 10 kDa molecular weight cut-off, dialyzed overnight at 4° C. against phosphate-buffered saline (137 mM sodium chloride, 2.7 mM potassium chloride, 7.7 mM disodium hydrogen phosphate, 1.47 mM potassium dihydrogen phosphate, pH 7.4; referred to as PBS), concentrated by using a 10 kDa centrifuge tube (Millipore); and stored at -20° C. The recombinant LTIIb-B5 protein was identified by sodium dodecyl sulfate-polyacrylamide gel electrophoresis (SDS-PAGE) and western blotting assay.

### Sds-Page

The operation of SDS-PAGE was briefly described as follows. First, protein samples and SDS loading buffer (50 mM Tris-HCl, pH 6.8; 100 mM dithiothreitol (DTT); 2 % SDS; 0.1% bromophenol blue; and 10% glycerin) were mixed in a ratio of 3:1 and then were boiled for 10 minutes.

At the same time, an electrophoresis gel, which comprises: a separating gel (taking a 12% separating gel as an example: 2.5 mL of 1 M Tris, pH 8.8, 3.3 mL of deionized water, 4 mL of 30% acrylamide mix, 0.1 mL of 10% SDS, 0.1 mL of 10% ammonium persulfate (APS); 0.01 mL of tetramethylethylenediamine (TEMED)), and a stacking gel (taking a 5% stacking gel as an example: 0.63 mL of 1 M Tris, pH 6.8, 3.4 mL of deionized water; 0.83 mL of 30% acrylamide mix, 0.05 mL of 10% SDS, 0.05 mL of 10% APS, 0.005 mL TEMED), was prepared.

Protein focusing within the stacking gel was performed at 80 V, and protein separating within the separating gel was performed at 140 V; wherein, the time of electrophoresis depended on the molecular weight of loaded proteins. The electrophoresis gel was then dyed with coomassie brilliant blue dye (0.1% coomassie R250, 10% acetic acid, 50% methanol) for 1 hour, and then was decolorized with a decolorizing solution (10% acetic acid, 50% methanol).

### Western Blotting Assay

The operation of western blotting assay was briefly described as follows. In a transfer tank, the protein samples separated by SDS-PAGE on the electrophoresis gel were transferred onto a nitrocellulose membrane (NC membrane) at 135 V, and then the NC membrane containing the transferred proteins was soaked in 20 mL tris-buffered saline solution containing tween-20 with 5% skimmed milk (TBST solution; 50 mM Tris, 150 mM sodium chloride, 0.05% Tween-20), and then was shaken for at least 1 hour to block non-specific binding.

The NC membrane was then washed three times with TBST solution, added with the primary antibody diluted with TBST solution at 1:5000, and shaken at 4° C. for about 16 hours. After being washed three times with TBST solution, the NC membrane was then added with the secondary antibody, which was connected to horseradish peroxidase (HRP), diluted with TBST solution at 1:3000, and shaken at room temperature for 1 hour. After being washed three times with TBST solution, the NC membrane was then added with an enhanced chemiluminescence reagent (Western Lighting Plus ECL; PerkinElmer) and western lighting plus ECL for one minute (PerkinElmer) to generate luminescence signals, which were visualized after exposure to Medical X-ray Film (Fujifilm).

### Experimental Mice Nasal Immunization

In one embodiment of the present invention, the immunization experiments of mucosal vaccination were carried out with six-to-eight-week BALB/c female mice, and each mouse was intranasally injected with PBS solution only or vaccine compositions containing recombinant proteins in PBS solution for intranasal mucosal immunization. In order to facilitate the nasal injection, the mice were anesthetized with isoflurane (Panion & BF Biotech Inc.) for general inhalation anesthesia, and then the vaccine compositions were dripped into the nasal cavities of the mice. Mice in each group were given three doses of immunization with an interval of about three weeks. All experiments were conducted in accordance with the guidelines of the Laboratory Animal Center of the National Tsing Hua University (NTHU). Animal use protocols are reviewed and approved by the NTHU Institutional Animal Care and Use Committee (approval no. 109047).

### Experimental Mice Serum Samples Collection

The mice were intranasally immunized by the aforementioned method, and serum samples of each mouse were collected at the 2^{nd} week after each dose of immunization. Mice were warmed up by ultra-red lamp and thermal blanket for 10 minutes before sampling. After being sterilized with 70% ethanol, the mouse lateral tail vein was cut with a surgical blade, and around 500 µL of blood was collected. The whole blood was left undisturbed at room temperature for 2 hours allowing the blood to clot. The clot was removed by centrifuging twice at 800 g for 15 minutes and serum was immediately transferred to new tubes. The serum was incubated at 56° C. for 30 min to inactivate complements. After cooling down to room temperature, the serum was apportioned and stored at -20° C.

### Experimental Mice CLNs and SPLs Samples Collection

The mice were intranasally immunized by the aforementioned method, and all mice were sacrificed at the 3^{rd} week after the last immunization. Mice cervical lymph nodes (CLNs) and splenocytes (SPLs) were collected for further evaluations. 5×10⁶ SPLs or 1×10⁶ CLNs were seeded in 96-well culture plates and stimulated by 1 µg/mL RBD protein in the total volume of 250 µL complete RPMI medium (Roswell Park Memorial Institute; Thermo Scientific™). After 72 hours of induction, the culture medium was collected and centrifuged at 1500 rpm for 5 minutes. The supernatants were apportioned and stored at -20° C.

### Experimental Mice BALFs Samples Collection

The mice were intranasally immunized by the aforementioned method, and all mice were sacrificed at the 3^{rd} week after the last immunization. The trachea of mice was exposed surgically and inserted with a syringe. 800 µL of PBS solution was injected into the bronchus and aspirated by the syringe. The Broncho-alveolar lavage fluid (BALFs) was centrifuged at 800 g for 15 minutes. The supernatants were transferred to new tubes and stored at -20° C.

### Preparation of SARS-CoV-2 Pseudovirus

The preparation method of the SARS-CoV-2 pseudovirus was briefly as follows. HEK-293A cells were seeded in a 10-cm dish with the amount of 3×10⁶ cells/plate, and were incubated overnight at 37° C. with 5% carbon dioxide. The pcDNA(TM)3.1(-) plasmid expressing the luminescence reporter gene (firefly luciferase) and full-length spike protein gene of SARS-CoV-2 (Wuhan-Hu-1, Alpha, Beta, or Delta), the pLAS2W.FLuc.Ppur reporter plasmid (HIV virus backbone), and HIV gag-pol plasmid (pCMVΔR8.91) were all co-transfected into the HEK293T cells by TransIT-LT1 transfection reagent (Mirus Bio). The culture medium was collected and concentrated 72 hours after transfection and stored at -20° C. Additionally, the titer of pseudovirus could be evaluated by detecting the luciferase activity in the HEK293 cells which were stably expressed ACE2 and infected with the SARS-CoV-2 pseudovirus.

### Determination of Cytokine Levels

The CLNs and the SPLs of mice were ground, and then were filtered with a cell strainer (Falcon). Red blood cells were removed with RBC lysis buffer (Invitrogen). Next, the obtained SPLs were cultured in a 96-well culture plate with the amount of 5×10⁵ cells/well, and the obtained CLNs were cultured in a 96-well culture plate with the amount of 1×10⁵ cells/well. The cells were stimulated with 1 µg/mL RBD protein and cultured at 37° C. with 5% carbon dioxide for 72 hours. The amount of IFN-y, IL-5, and IL-17A secreted by T helper 1 cells (Th1 cells), T helper 2 cells (Th2 cells), and T helper 17 cells (Th17 cells) in the culture medium was analyzed by ELISA.

### Experimental Hamster Nasal Immunization

In one embodiment of the present invention, the immunization experiments of mucosal vaccination were carried out with six-to-eight-week female golden Syrian hamsters, and each hamster was intranasally injected with vaccine compositions containing recombinant proteins for intranasal mucosal immunization. In order to facilitate the nasal injection, the hamsters were anesthetized by intraperitoneal injection of Zoletil/Xylazine (Rompun) (20-40 mg/kg Zoletil + 5-10 mg/kg Xylazine) and inhalation of isoflurane (Panion & BF Biotech Inc.), and then the vaccine compositions were dripped into the nasal cavities of the hamsters. Hamsters in each group were given three doses of immunization with an interval of about three weeks.

### Experimental Hamster Serum Samples Collection

The hamsters were intranasally immunized by the aforementioned method, and serum samples of each hamster were collected at the 2^{nd} week after each dose of immunization. The hamster was anesthetized as described above before sampling. The hamster was properly restrained in the hand and the gingival papillae region was exposed. The needle was inserted about 2 mm into the gingiva and collected approximately 100 µL of blood from the gingival vein. The whole blood was left undisturbed at room temperature for 2 hours allowing the blood to clot. The clot was removed by centrifuging twice at 800 g for 15 minutes and serum was immediately transferred to new tubes. The serum was incubated at 56° C. for 30 min to inactivate complements. After cooling down to room temperature, the serum was apportioned and stored at -20° C.

### Example 1

### The Preparation of the Antigen Fusion Protein

In the SARS-CoV-2 mucosal vaccine composition of the present invention, the protein subunit was used as the antigen, and in one embodiment of the present invention, the receptor-binding domain (RBD) of the spike protein (also known as S protein) was used as an example of SARS-CoV-2 antigen to illustrate the preparation method of the antigen protein subunit in the SARS-CoV-2 mucosal vaccine composition of the present invention.

In order to overcome the problem that when protein subunits were used as antigens in vaccines, adjuvants were necessary to effectively induce immune responses, the adjuvants were directly fused with antigens to form the mucosal vaccine composition of the present invention with a built-in adjuvant. In the embodiment of the present invention, the A subunit of heat-labile enterotoxin (LT) from Escherichia coli Type IIb (E. coli type IIb) was used as an example of an embedded adjuvant.

Therefore, the protein subunit antigen in all of the embodiments herein was a fusion protein consisted of the receptor-binding domain of the spike protein of SARS-CoV-2 and Type IIb heat-labile enterotoxin A subunit from E. coli, hereinafter referred to as RBD -LTIIbA fusion protein.

In the embodiment of the present invention, in order to prevent RBD and LTIIbA from interfering with each other and affecting the original structure or activity of antigen and adjuvant when they were formed together in the RBD-LTIIbA fusion protein, a short amino acid segment could be used to connect and separate RBD and LTIIbA, so as to separate RBD and LTIIbA in the three-dimensional structure and reduce the mutual interference during protein folding. The short amino acid segment may contain glycine (Gly) and serine (Ser), such as a GS linker containing Gly-Gly-Ser-Gly-Gly-Gly-Ser-Gly (SEQ ID NO:).

Further, in order to facilitate subsequent separation of the RBD-LTIIbA fusion protein in an in vitro protein expression system, an additional signal peptide segment of an envelope glycoprotein could be added to the N-terminus region of the RBD-LTIIbA fusion protein to make the RBD-LTIIbA fusion protein secreted out of host cells of the protein expression system. In the embodiment of the present invention, the signal peptide segment of the envelope glycoprotein of the Autographa californica nucleopolyhedrovirus (AcMNPV) (sequence number in the open database: AAA72759.1) (SEQ ID NO:3) was used. In addition, in order to facilitate subsequent purification of the RBD-LTIIbA fusion protein, an additional histidine tag (His tag) could be added in front of the signal peptide segment or between the signal peptide segment and the RBD-LTIIbA fusion protein to make the RBD-LTIIbA fusion protein purified easily by nickel ion chelating resin (TOSOH).

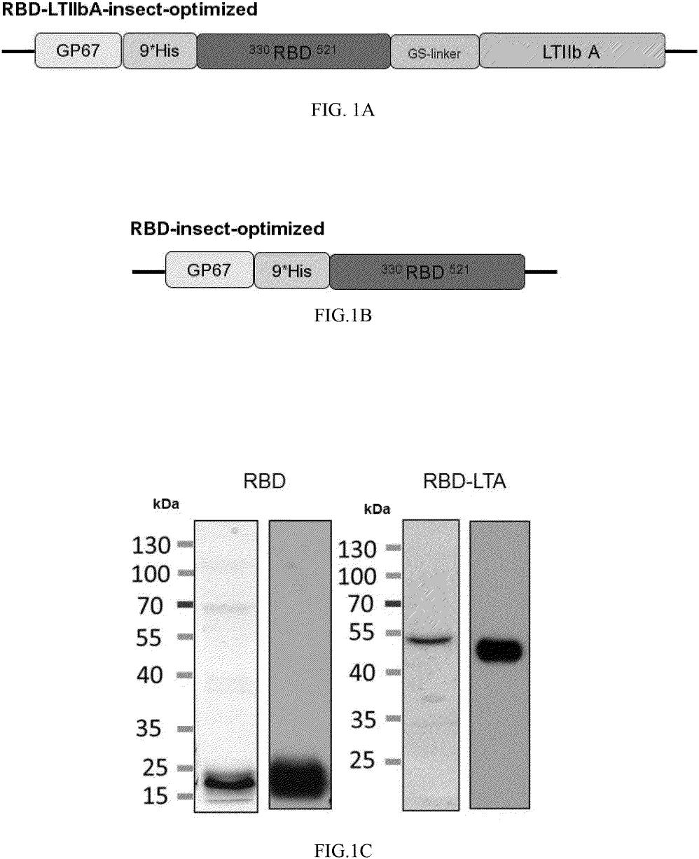

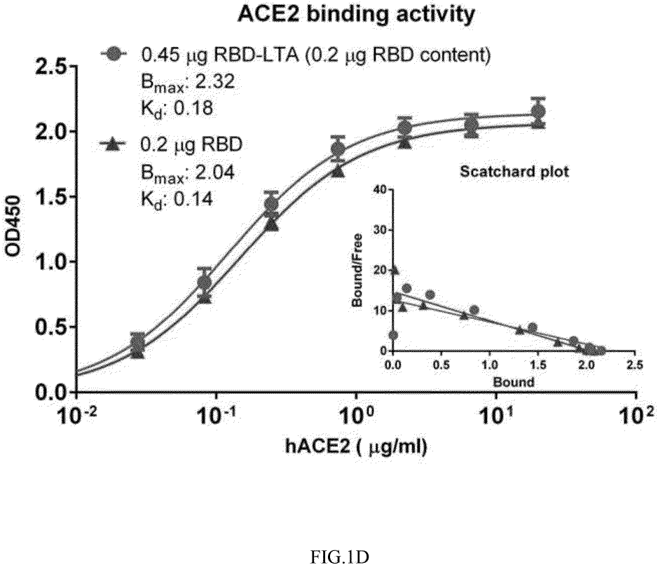

The preparation method of the RBD-LTIIbA fusion protein of the present invention was briefly described in detail as follows. First, a DNA construct was constructed. Please refer to FIG. 1A showing a schematic view of the DNA construct of the RBD-LTIIbA fusion protein of the present invention, which contained in order from the 5′end to the 3′end: the nucleic acid sequence (SEQ ID NO:4) corresponding to the signal peptide segment of the envelope glycoprotein (GP67 gene) (represented as GP67); the nucleic acid sequence (SEQ ID NO:5) corresponding to the histidine tag consisted of nine consecutive histidines (represented as 9*His); the nucleic acid sequence corresponding to the receptor-binding domain of the spike protein of SARS-CoV-2 (represented as RBD); the nucleic acid sequence corresponding to the short peptide segment of the GS linker molecule (represented as GS-linker); and the nucleic acid sequence corresponding to the Type IIb heat-labile enterotoxin A subunit from E.coli (represented as LTIIb A); wherein, each nucleic acid sequence was commissioned to a gene synthesis company (such as GenScript) to optimize and synthesize.

The nucleic acid sequence of the receptor-binding domain and the Type IIb heat-labile enterotoxin A subunit from E. coli were obtained from the public database; wherein, the nucleic acid sequence of the receptor-binding domain (SEQ ID NO: 6) was from the gene sequence corresponding to the 330-521 amino acid sequence of the spike protein of SARS-CoV-2 (Wuhan-Hu-1, accession number: MN908947.3), and the codons were optimized to be suitable for insect cells; the nucleic acid sequence of the Type IIb heat-labile enterotoxin A subunit from E.coli (accession number: P43528.2) (SEQ ID NO: 7) was also optimized to be suitable for insect cells, and the nucleic acid sequence used herein could have more than 90% sequence identity with SEQ ID NO:7.

In addition, in order to further compare the differences between the vaccine composition containing the RBD-LTIIbA fusion protein of the present invention with a built-in adjuvant and the vaccine composition only containing the RBD protein in the efficacy of inducing immune responses, the other DNA construct was constructed. Please refer to FIG. 1B showing a schematic view of the DNA construct of the RBD protein, which contained in order from the 5′end to the 3′end: the nucleic acid sequence (SEQ ID NO:4) corresponding to the signal peptide segment of the envelope glycoprotein (GP67 gene) (represented as GP67); the nucleic acid sequence (SEQ ID NO:5) corresponding to the histidine tag consisted of nine consecutive histidines (represented as 9*His); and the nucleic acid sequence corresponding to the receptor-binding domain of the spike protein of SARS-CoV-2 (represented as RBD).

Next, the two DNA constructs were respectively cloned into pFastBac1 vectors (Invitrogen), and transformed into competent cells (E.coli Top 10 strain, Invitrogen) to be amplified. Hereinafter, the two DNA constructs cloned into the pFastBac1 vector were called pFastBac-RBD-LTIIbA and pFastBac1-RBD, respectively. Next, pFastBacl-RBD or pFastBac1-RBD-LTA were respectively transformed into DH10Bac E. coli to generate gene-harbored bacmids (Bacmid-RBD, Bacmid-RBD-LTA). The Bac-to-Bac® Baculovirus Expression System (Invitrogen) was used to express the RBD-LTIIbA fusion protein and the RBD protein through the Bacmid-RBD-LTA and Bacmid-RBD, respectively. In short, a recombinant baculovirus with the Bacmid-RBD-LTA or the Bacmid-RBD was produced, and then insect cells, such as Sf9 cells were infected with the recombinant baculovirus so that the insect cells could use the recombinant baculovirus to expresses the RBD-LTIIbA fusion protein of the present invention and the RBD protein.

The detailed preparation method of expressing protein was as follows. First, 1 ng of the pFastBac-RBD-LTIIbA or pFastBacl-RBD wers well mixed with 90 µL of competent cells (E.coli DH10 Bac strain, Invitrogen) suitable for the Bac-to-Bac® Baculovirus Expression System. The mixture was put on ice for 30 minutes, and in order to transform the vectors into the competent cells, the mixture was then put in a 42° C. environment for heat shock for 45 seconds and then stood on ice for another 2 minutes. Next, 900 µL of bacterial culture medium (such as LB broth) was added into the competent cells, and then the competent cells were incubated in a 37° C. incubator for 4 hours. Then, 10 µL of the culture medium was taken out and spread evenly on a LB culture plate with 50 µg/mL kanamycin, 7 µg/mL gentamicin, 10 µg/mL tetracycline, 100 µg/mL X-gal, and 40 µg/mL isopropyl-P-D-thiogalactin glycosides to select competent cells which have successfully transformed into the pFastBac-RBD-LTIIbA or pFastBac1-RBD, and such competent cells were cultured with conventional methods to amplify the genes inside and to make the vector recombine with the parental bacmids in the competent cells to form a gene-harbored bacmid, which was hereinafter referred to as Bacmid-RBD-LTIIbA or Bacmid-RBD.

The two gene-harbored bacmid, Bacmid-RBD-LTIIbA and Bacmid-RBD, were purified from the competent cells, and 5 µg of the gene-harbored bacmid were mixed together with 16 µL of turbofect transfection reagent (Thermo Scientific™) and OPTI-MEM culture medium (Thermo Scientific™), and then were stood for 15 minutes until the gene-harbored bacmid and the transfection reagents were completely mixed. The mixture was added to the Sf9 cells which have attached to the culture plate, and the cells were incubated in a 28° C. constant temperature incubator for 6 hours for transfection, and the culture medium was exchanged with 3 mL of fresh Sf900 II SFM Thermo Scientific™, 10% penicillin/streptomycin antibiotics) containing 5% FBS. After the cells were cultured in the 28° C. constant temperature incubator for 7 days, recombinant baculovirus (rBVs) containing the gene-harbored bacmid, Bacmid-RBD-LTIIbA or Bacmid-RBD, in the supernatant of the cell culture medium was collected. The gene-harbored bacmid for subsequent protein expressio was collected and amplified in the Sf9 cells for three passages.

Next, after being collected, the recombinant baculovirus containing the Bacmid-RBD-LTIIbA or the Bacmid-RBD was used to infect 1.8×10⁶ cells/mL of Sf9 cells, and the Sf9 cells were cultured at 28° C. in suitable spinner flasks for 96 hours to express the RBD-LTIIbA fusion protein or the RBD protein in the two vectors, the Bacmid-RBD-LTIIbA and the Bacmid-RBD, through the recombinant baculovirus. The RBD-LTIIbA fusion protein and the RBD protein were secreted into the cell culture medium, and thus the culture medium of the Sf9 cells was collected to obtain the RBD-LTIIbA fusion protein of the present invention or the RBD protein.

Therefore, for the subsequent protein purification, the collected culture medium was centrifuged at 3,000 rpm for 10 minutes and 9,000 rpm for 5 minutes at 4° C. to remove suspended Sf9 cells twice. After being concentrated by a filtration device with a molecular weight cut-off of 30 kDa or 10 kDa, the solution was centrifuged at 9,000 rpm for 10 minutes at 4° C. to remove the precipitate produced during the concentration. The concentrated solution was adjusted to pH 7.4 with Tris buffer solution (pH 8.0) and then centrifuged at 10,000 rpm for 5 minutes at 4° C. to remove the precipitate produced during the adjustment process. The solution was then filtered with a 0.45 µm filter membrane and was mixed with nickel ion chelating resin (TOSOH) overnight at 4° C. Then, 30-40% buffer solution B was used to elute the RBD-LTIIbA fusion protein of the present invention and the RBD protein. The purification fraction of the RBD-LTIIbA fusion protein and the RBD protein was concentrated with a 30 kDa or 10 kDa concentrated centrifuge tube (Millipore) into PBS solution and stored at 4° C. A small amount of each sample was taken and analyzed by SDS-PAGE and Western blotting assay to confirm the size and identity of the RBD-LTIIbA fusion protein and the RBD protein.

The detailed analysis methods of SDS-PAGE and western blotting assay of the purified RBD-LTIIbA fusion protein and RBD protein samples were as follows. First, 15 µL of each sample was taken and mixed with 5 µL loading buffer solution containing SDS, and then was boiled for 10 minutes. The samples were then loaded into an SDS-PAGE gel (10% separating gel) and were performed focusing at 80 V for 20 minutes. After the proteins of each sample were transferred from the stacking gel to a separating gel, the voltage was adjusted to 140 V for about 2.5-3 hours to separate proteins of different sizes. After the electrophoresis, the gel was dyed and decolorized by the aforementioned method. The results were shown on the left side of FIG. 1C. The RBD protein sample contained a major protein band at about 20 kDa, which was equivalent to the molecular weight of the RBD protein (about 22 kDa). The RBD-LTIIbA fusion protein of the present invention sample contained a major protein band between 40-55 kDa, which was equivalent to the molecular weight that the RBD-LTIIbA fusion protein should be, i.e. the total molecular weight (about 50 kDa) of the RBD protein and the Type IIb heat-labile enterotoxin A subunit from E. coli (about 28 kDa).

Next, western blotting assay was performed to confirm whether the gel of SDS-PAGE contained the RBD-LTIIbA fusion protein of the present invention and the RBD protein. First, the SDS-PAGE gel was placed in the transfer tank with an NC membrane at 135 V for 40 minutes to transfer proteins from the gel to the NC membrane. Anti-RBD antibody (GTX135385; Genetex; dilution in 1:5000 with TBST solution) was used as the primary antibody, and HRP-conjugated anti-rabbit antibody (dilution in 1:10000) was used as the secondary antibody. The operation of western blotting assay was performed by the aforementioned method. The results were shown as the left side of FIG. 1C. The RBD protein signal could be detected at the aforementioned major protein band at about 20 kDa. The RBD-LTIIbA fusion protein signal could be detected at the aforementioned major protein band between 40-55 kDa, and the RBD protein signal could also be detected. The results indicated that after purification, the RBD-LTIIbA fusion protein of the present invention and the RBD protein samples indeed contain the RBD-LTIIbA fusion protein and the RBD protein, respectively.

In the embodiment of the present invention, in order to further confirm whether the produced RBD protein and the RBD protein in the RBD-LTIIbA fusion protein of the present invention still contained the functions of the original RBD protein, the enzyme-linked immunosorbent assay (ELISA) was used to detect the binding affinity between the recombinant RBD proteins and angiotensin-converting enzyme 2 (hereinafter referred to as ACE2) for analysis. The detailed method was as follows. First, the recombinant RBD protein at the concentration of 0.2 µg/well and the RBD-LTIIbA fusion protein of the present invention at the concentration of 0.45 µg/well (containing about 0.2 µg/well of RBD protein) was coated onto two different 96-well ELISA plates (Thermo) in 0.05 M Carbonate-Bicarbonate Buffer (i.e. coating buffer) overnight at 4° C. The coating buffer was aspirated and each well was washed with 300 µL of PBS solution containing 0.05% Tween 20 (hereinafter referred to as PBST solution) three times to wash off excess recombinant proteins. 150 µL of blocking buffer (1% bovine serum albumin (BSA) in PBST solution) was added into each well at room temperature for 2 hours to block non-specific binding. The blocking buffer was aspirated and each well was washed with PBST solution three times to wash off excess blocking buffer. Next, ACE protein (Z03516; GenScript) was serially diluted with a dilution buffer solution (1% BSA and 0.05% Tween 20 in PBS) starting from 10 µg/mL. The serial dilutions of ACE2 protein were added into the 96-well culture plate at room temperature for 1 hour to allow ACE2 proteins in the samples combined with the RBD protein or the RBD-LTIIbA fusion protein, and then each well was washed with PBST solution three times to wash off excess ACE2 proteins. Then, 100 µL of anti-ACE2 antibody (diluted in 1:1000 with PBST solution) was added into each well of the 96-well culture plate at room temperature for 1 hour, and then each well was washed three times with PBST solution to remove excess anti-ACE2 antibody. Next, 100 µL of anti-rabbit-HRP antibody (diluted in 1:10000 with PBST solution) was added into each well of the 96-well culture plate at room temperature in the dark for 1 hour to identify the anti-ACE2 antibody. Each well was washed three times with PBST solution to remove excess antibodies. Finally, 100 µL of the substrate 3,3′,5,5′-Tetramethyl- benzidine (TMB, BioLegend) of HRP was added to each well of the 96-well culture plate and incubated in the dark for 15 minutes. The reaction was stopped with 100 µL of 2 N H₂SO₄. The optical density at 450 nm was then measured by an ELISA analyzer (TECAN). Each absorbance was plotted with the corresponding concentration of ACE2 protein to determine the binding affinity between the recombinant RBD proteins and ACE2 protein. In order to more directly observe the binding relationship between the substrate and the receptor, the Scatchard plot was further provided. The equation was: B/F=Bmax/Kd-B/Kd; the Y-axis was B/Y; the X-axis was B; the intercept of the Y-axis was Bmax/Kd; the intercept of the X-axis was Bmax. B was the binding substrate (reads of absorbance); F was the concentration of free receptor; Bmax was the maximum binding strength of the substrate (absorbance at 450 nm, that is, the signal strength of ACE2), which was used to describe the binding strength when all RBD proteins fully bound with ACE2; Kd was the dissociation constant, which was used to describe the constant of binding-separating of the RBD protein and ACE2, and the larger the constant was, the lower the binding degree was; wherein, with conventional calculation software, Bmax and Kd could be calculated from the curve of absorbance over the corresponding concentration of ACE2 protein.

FIG. 1D shows the testing results of the binding affinity of the recombinant RBD protein and the RBD protein in the RBD-LTIIbA fusion protein of the present invention to ACE2 protein. As shown in FIG. 1D, with the increase in the concentration of ACE2 protein, both of the binding amount of the recombinant RBD protein and the recombinant RBD-LTIIbA fusion protein of the present invention to ACE2 protein increased, indicating that both the recombinant RBD protein and the RBD-LTIIbA fusion protein of the present invention maintained the function of the original RBD protein to bind ACE2. In any concentration of ACE2 protein, the binding affinity between the RBD-LTIIbA fusion protein of the present invention and ACE2 protein was equivalent to the binding affinity between the recombinant RBD protein and ACE2 protein. The results indicated that the structure of the RBD-LTIIbA fusion protein of the present invention would not affect the binding stability between the RBD protein and ACE2 because of the fused LTIIbA.

### Example 2

### The SARS-CoV-2 Mucosal Vaccine Composition Induces Systemic and Mucosal Immunity in Mice

In one embodiment of the present invention, in order to test whether the SARS-CoV-2 mucosal vaccine composition of the present invention could effectively induce systemic immune responses and mucosal immune responses against SARS-CoV-2 in mammals without additional adjuvants, the vaccine composition containing the RBD-LTIIbA fusion protein of the present invention was injected into the nasal cavity of mice, and after a period of time, serum and BALFs of mice were collected to test whether the serum and the BALFs contained antibodies against SARS-CoV-2, and the vaccine composition containing the RBD protein and different additional adjuvants was used as comparison groups.

First, PBS solution was used as a diluent to formulate the following six groups into a 30 µL solution dosage form of vaccine composition: (1) the negative control group containing only PBS solution (represented as PBS); (2) the comparison group containing 20 µg of the RBD protein (represented as RBD); (3) the comparison group containing 20 µg of the RBD protein and 5 µg of LTIIb-B5 protein (represented as RBD+LTIIbB); (4) the experimental group containing 45 µg of the RBD-LTIIbA fusion protein of the present invention (the content of the RBD protein was 20 µg) (represented as RBD-LTIIbA); (5) the comparison group containing 20 µg of the RBD protein and 2 µg of polyinosinic acid-polycytidylic acid (poly(I:C)), wherein poly(I:C) was known as an effective mucosal vaccine adjuvant (represented as RBD+poly(I:C)); and (6) the positive control group containing 10⁸ PFU of the adenovirus vector, on which the spike protein of SARS-CoV-2 was expressed (represented as Ad-S). Next, the six groups of vaccine composition were used to immunize female BALB/c mice by nasal injection at the 0, 3^{rd}, and 6^{th} week, respectively, with at least five mice in each group. Serum samples of each group of mice were collected at the 2^{nd} week after each immunization, and the mice were sacrificed at the 3^{rd} week after the last immunization to collect BALFs samples of each group of mice to test whether there were antibodies against SARS-CoV-2 inside serum and BALFs of the mice.

Then, the serum and BALFs samples collected from each group of mice were detected by ELISA to confirm there were IgG and IgA antibodies against SARS-CoV-2, so as to determine whether the systemic immune responses and mucosal immune responses of mice were successfully elicited, respectively. First, 100 µL of RBD protein with the concentration of 0.2 µg/well was coated onto 96-well ELISA plate (Thermo) in 0.05 M Carbonate-Bicarbonate Buffer (i.e. coating buffer) overnight at 4° C. The coating buffer was aspirated and each well was washed with 300 µL of PBST solution three times to wash off excess RBD proteins. The blocking buffer was added into each well at room temperature for 2 hours to block non-specific binding. The blocking buffer was aspirated and each well was washed with PBST solution three times to wash off excess blocking buffer. Then, the serum and BALFs samples of each group of mice were serially 2-fold diluted with a dilution buffer solution starting from the original to 10000-fold-diluted solution, and 100 µL of the serially diluted serum or BALFs was added into the 96-well culture plate at room temperature for 1 hour to allow specific antibodies in the serum or BALFs samples combined with the RBD protein. Herein, two separated serial dilutions of each serum sample had to be prepared for detecting IgG antibodies and IgA antibodies, respectively. Each well was washed with PBST solution three times to wash off excess serum or BALFs. Next, 100 µL of anti-mouse IgG-HRP antibody (diluted in 1:30000) or anti-mouse IgA-HRP antibody (diluted in 1:50000) was added into each well of the 96-well culture plate at room temperature in the dark for 1 hour. Each well was washed three times with PBST solution to remove excess antibodies. Finally, 100 µL of the substrate TMB of HRP was added to each well of the 96-well culture plate and incubated in the dark for 15 minutes, and then the reaction was stopped with 2 N H₂SO₄. The optical density at 450 nm was then measured by an ELISA analyzer (TECAN). The end-point titer of the anti-RBD IgG antibodies or the anti-RBD IgA antibodies was calculated by a final serial dilution higher than 0.2 in the optical density value, and if the optical density value of the highest dilution exceeded 0.2 or the optical density value of the lowest dilution was less than 0.2, the experiment should be repeated at higher or lower dilution.

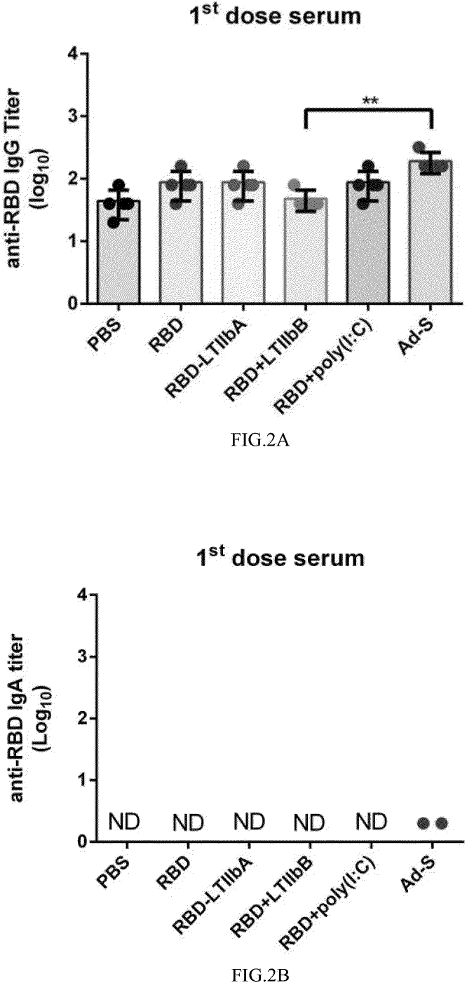

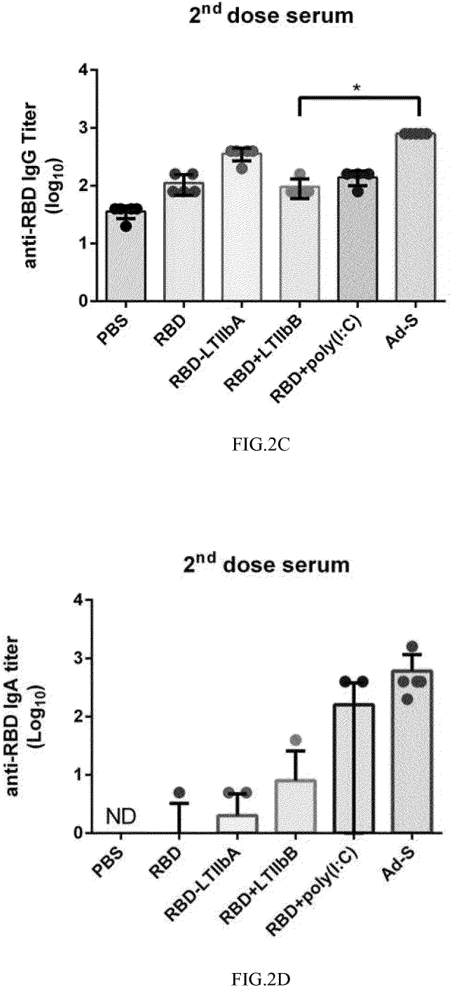

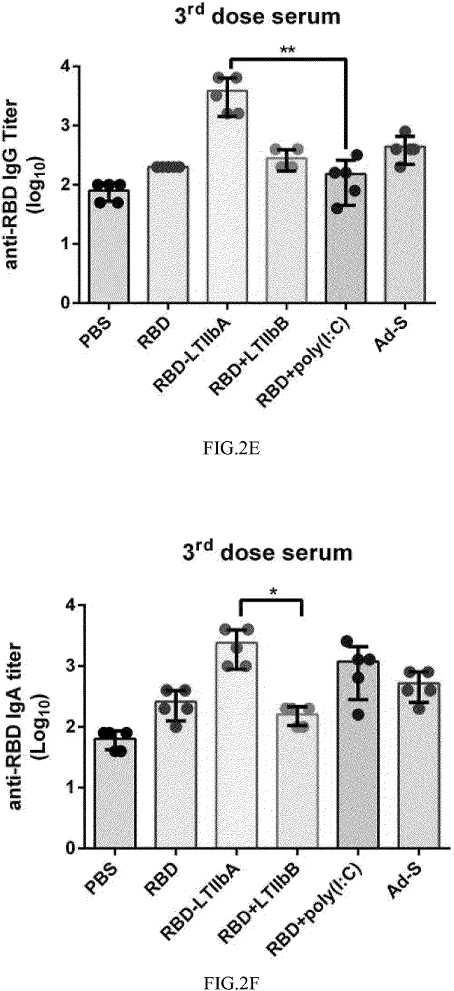

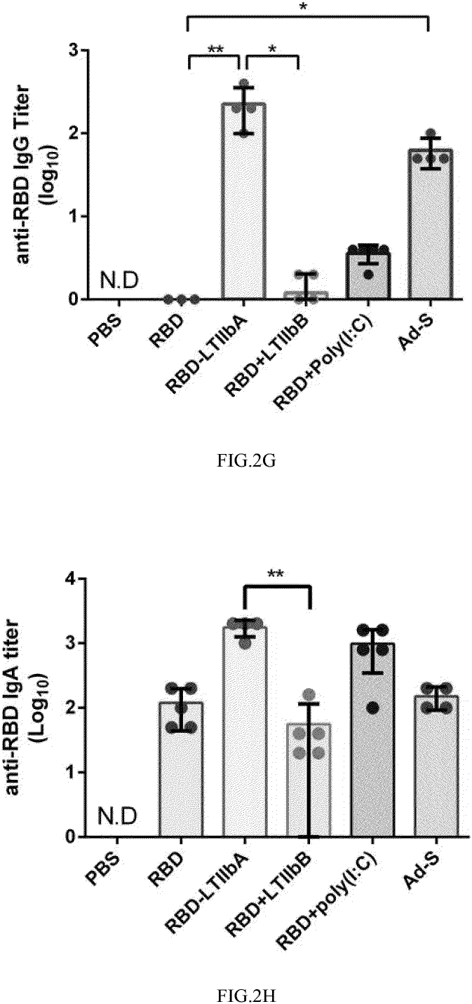

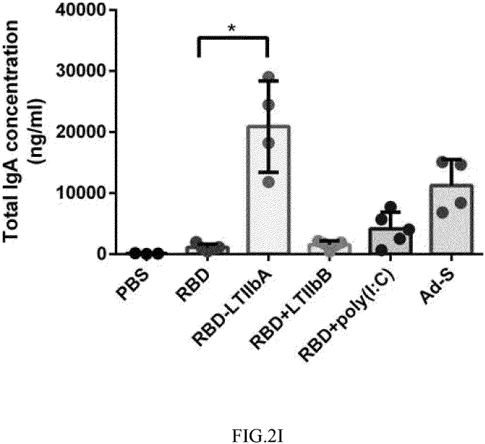

The titers of anti-RBD IgG antibodies and IgA antibodies in the serum of mice after being intranasally immunized with the 1^{st} dose of the mucosal vaccine composition containing the RBD-LTIIbA fusion protein of the present invention alone or the RBD protein together with different additional adjuvants were shown as FIG. 2A and FIG. 2B, respectively. Each data and the average thereof were shown in the FIGs. * represented p <0.05; ** represented p <0.01; *** represented p <0.001; and **** represented p <0.0001. As shown in FIG. 2A, after being immunized with the 1^{st} dose of the mucosal vaccine composition, the titer of anti-RBD IgG antibodies induced by the RBD-LTIIbA fusion protein of the present invention alone was equivalent to the titer of anti-RBD IgG antibodies induced by the RBD protein alone or together with different additional adjuvants, and was close to the result of the negative control group, and only Ad-S in the positive control group could elicit a slightly higher titer of anti-RBD IgG antibodies, indicating that being immunized with the 1^{st} dose of the vaccine composition of each group cannot effectively induce systemic immune responses of the subjects. As shown in FIG. 2B, after being immunized with the 1^{st} dose of the mucosal vaccine composition, the titer of anti-RBD IgA antibodies could not be effectively induced by any groups of mucosal vaccine composition, indicating that being immunized with the 1^{st} dose of each vaccine composition could not effectively induce mucosal immune responses of the subjects.

The titers of anti-RBD IgG antibodies and IgA antibodies in the serum of mice after being intranasally immunized with the 2^{nd} dose of the mucosal vaccine composition were shown as FIG. 2C and FIG. 2D, respectively. Each data and the average thereof were shown in the FIGs. * represented p <0.05; ** represented p <0.01; *** represented p <0.001; and **** represented p <0.0001. As shown in FIG. 2C, the titer of anti-RBD IgG antibodies induced by the RBD-LTIIbA fusion protein of the present invention alone was higher than the titer of anti-RBD IgG antibodies induced by the RBD protein alone or together with all different additional adjuvants, and was close to the result of the positive control group that provided high ability to induce the production of anti-RBD IgG antibodies, indicating that being immunized with only two intranasal doses of the mucosal vaccine composition of the present invention without any additional adjuvants were required to effectively induce systemic immune responses against SARS-CoV-2. As shown in FIG. 2D, after being immunized with the 2^{nd} dose of the mucosal vaccine composition, Ad-S in the positive control group induced the highest titer of anti-RBD IgA antibodies, indicating that being immunized with two doses of Ad-S could induce the highest mucosal immune responses in the subjects.

The titers of anti-RBD IgG antibodies and IgA antibodies in the serum of mice after being intranasally immunized with the 3^{rd} dose of the mucosal vaccine composition were shown as FIG. 2E and FIG. 2F, respectively. Each data and the average thereof were shown in the FIGs. * represented p <0.05; ** represented p <0.01; *** represented p <0.001; and **** represented p <0.0001. As shown in FIG. 2E, after being immunized with the 3^{rd} dose of the mucosal vaccine composition, the titer of anti-RBD IgG antibodies induced by the RBD-LTIIbA fusion protein of the present invention alone was higher than the titer of anti-RBD IgG antibodies induced by the RBD protein alone or together with all different additional adjuvants, and was even much higher than the titer of anti-RBD IgG antibodies induced by Ad-S of the positive control group. As shown in FIG. 2F, after being immunized with the 3^{rd} dose of the mucosal vaccine composition, the titer of anti-RBD IgA antibodies induced by the RBD-LTIIbA fusion protein of the present invention alone was higher than the titer of anti-RBD IgA antibodies induced by the RBD protein alone or together with all different additional adjuvants, and was also higher than the titer of anti-RBD IgA antibodies induced by Ad-S of the positive control group. The results indicated that being intranasal immunized with the mucosal vaccine composition of the present invention could not only effectively induce systemic immune responses against SARS-CoV-2, but also could effectively induce mucosal immune responses without any additional adjuvants, and the effects were significantly better than the positive control group.

The titers of anti-RBD IgG antibodies and IgA antibodies in the BALFs of mice after being intranasally immunized with the 3^{rd} dose of the mucosal vaccine composition were shown as FIG. 2G and FIG. 2H, respectively. Each data and the average thereof were shown in the FIGs. * represented p <0.05; ** represented p <0.01; *** represented p <0.001; and **** represented p <0.0001. As shown in FIG. 2G, after being immunized with the 3^{rd} dose of the mucosal vaccine composition, the titer of anti-RBD IgG antibodies induced by the RBD-LTIIbA fusion protein of the present invention alone was higher than the titer of anti-RBD IgG antibodies induced by the RBD protein alone or together with all different additional adjuvants, and was even much higher than the titer of anti-RBD IgG antibodies induced by Ad-S of the positive control group. As shown in FIG. 2H, after being immunized with the 3^{rd} dose of the mucosal vaccine composition, the titer of anti-RBD IgA antibodies induced by the RBD-LTIIbA fusion protein of the present invention alone was higher than the titer of anti-RBD IgA antibodies induced by the RBD protein alone or together with all different additional adjuvants, and was also higher than the titer of anti-RBD IgA antibodies induced by Ad-S of the positive control group. The results indicated that being intranasal immunized with the mucosal vaccine composition of the present invention without additional adjuvants could effectively induce mucosal immune responses in the respiratory tract to produce antigen-specific IgG antibodies and IgA antibodies against SARS-CoV-2, and directly increase the ability of the respiratory tract to resist SARS-CoV-2, and the effects were significantly better than the positive control group.

In order to further understand the ability of the SARS-CoV-2 mucosal vaccine composition of the present invention to induce overall mucosal immune responses, the aforementioned BALFs was detected by ELISA to confirm the concentration of total IgA antibodies. The following procedures for detecting the concentration of total IgA were referred to the manufacturer’s instructions (Invitrogen). First, 100 µL of the capture antibodies were coated onto a 96-well culture plate in the coating buffer overnight at 4° C. The coating buffer was aspirated and each well was soaked with 300 µL of PBST solution for 1 minute three times to wash off excess capture antibodies. Then, 250 µL of the blocking buffer was added into each well at room temperature for 2 hours to block non-specific binding. The blocking buffer was aspirated and each well was washed with PBST solution three times to wash off excess blocking buffer. Next, the commercial standard and the BALFs of each group of mice were serially diluted, and then the serial dilutions were added into the 96-well culture plate at room temperature for 2-hour shaking to allow IgA antibodies inside to bind to the capture antibodies. The serial dilutions were aspirated and each well was washed with PBST solution four times. Next, 100 µL of the detection antibodies were added into each well at room temperature for 1-hour shaking. The detection antibodies were aspirated and each well was washed with PBST solution four times to wash off excess detection antibodies. Finally, 100 µL of TMB was added into each well and incubated in the dark for 15 minutes. The reaction was stopped with 100 µL of 2 N H₂SO₄. The optical density at 450 nm was then measured by the ELISA analyzer (TECAN). Each concentration of the standard and the corresponding absorbance were used to draw a standard curve for calculating the concentration of IgA antibody in BALFs of each group of mice.

The concentration of total IgA antibodies in the BALFs of mice after being intranasally immunized with the 3^{rd} dose of the mucosal vaccine composition was shown as FIG. 2I. Each data and the average thereof were shown in the FIG. * represented p <0.05; ** represented p <0.01; *** represented p <0.001; and **** represented p <0.0001. As shown in FIG. 2I, after being immunized with the 3^{rd} dose of the mucosal vaccine composition, the concentration of total IgA antibodies induced by the RBD-LTIIbA fusion protein of the present invention alone was higher than the concentration of total IgA antibodies induced by the RBD protein alone or together with all different additional adjuvants, and was even much higher than the concentration of total IgA antibodies induced by Ad-S of the positive control group. The results indicated that being intranasal immunized with the mucosal vaccine composition of the present invention without additional adjuvants could effectively induce overall mucosal immune responses in the respiratory tract to increase the ability of the respiratory tract to resist pathogens including SARS-CoV-2, so as to reduce the possibility of infection of SARS-CoV-2 through the respiratory tract of the subjects.

Together with all results of the embodiment, intranasal immunization with the mucosal vaccine composition of the present invention without additional adjuvants could significantly increase the amount of antigen-specific IgG and IgA antibodies in mammals, especially the amount of IgA antibodies in BALFs, and could also significantly increase the concentration of total IgA antibodies in the respiratory tract. Therefore, the vaccine composition containing the RBD-LTIIbA fusion protein of the present invention provided effects on eliciting systemic and mucosal immunity in mammals.

### Example 3

### The SARS-CoV-2 Mucosal Vaccine Composition Induces the Production of High Titer Neutralizing Antibodies

In one embodiment of the present invention, in order to test the titer of the neutralizing antibody induced by the SARS-CoV-2 mucosal vaccine composition of the present invention, the pseudovirus neutralization assay was used to test the efficacy of neutralizing antibodies produced by mice after being immunized with the SARS-CoV-2 mucosal vaccine composition of the present invention on neutralizing the infection of the original Wuhan-Hu-1 strain, the Alpha mutant strain (B.1.1.7), the Beta mutant strain (B.1351), and the Delta mutant strain (B.1.617.2) of SARS-CoV-2.

First, in the manner described in Example 2, the following six groups were formulated into a 30 µL solution dosage form of vaccine composition: (1) the negative control group containing only PBS solution (represented as PBS); (2) the comparison group containing 20 µg of the RBD protein (represented as RBD); (3) the comparison group containing 20 µg of the RBD protein and 5 µg of LTIIb-B5 protein (represented as RBD+LTIIbB); (4) the experimental group containing 45 µg of the RBD-LTIIbA fusion protein of the present invention (the content of the RBD protein was 20 µg) (represented as RBD-LTIIbA); (5) the comparison group containing 20 µg of the RBD protein and 2 µg of poly(I:C) (represented as RBD+poly(I:C)); and (6) the positive control group containing 10⁸ PFU of the adenovirus vector, on which the spike protein of SARS-CoV-2 was expressed (represented as Ad-S). Next, the mice were immunized by nasal injection as described in Example 2, and serum samples of each group of mice were collected for subsequent experiments.

Before testing the ability of the serum samples to neutralize infection of viruses, 2×10⁴ HEK-293T cells stably expressing human angiotensin-converting enzyme 2 (hACE2) were seeded in each well of a 96-well culture plate at 37° C. for one day; wherein, DMEM (Dulbecco’s Modified Eagle Medium) containing 1% fetal bovine serum (FBS) was used as the cell culture medium. The aforementioned serum samples were serially 2-fold diluted with Minimum Essential Media (MEM) containing 2% FBS from the 30-time-diluted solution. Each diluted serum was respectively incubated with 1,000 TU (transducing units) of the original Wuhan-Hu-1 strain, the Alpha mutant strain, the Beta mutant strain, and the Delta mutant strain of SARS-CoV-2 pseudovirus for 1 hour at 37° C. The mixture of the serum and the pseudovirus was then added into the aforementioned 96-well culture plate to infect the cells; wherein, the volume of the added mixture was equal to the volume of the culture medium in the culture plate, so the dilution fold would increase (starting at 640-fold). After the HEK-293T cells expressing hACE2 were infected at 37° C. for 1 hour, the culture medium was replaced with fresh DMEM containing 10% FBS. The HEK-293T cells were continuously cultured in the 37° C. incubator until 72 hours post-infection, during which time fresh cell culture medium should be replaced daily. The cells were lysed and subjected to luciferase assay (Promega Bright-GloTM Luciferase Assay System) to calculate the ability of serum from each group of mice to neutralize the viruses. The inhibition percentage of viral infection was calculated by “losing relative luciferase units (RLU) between serum and positive control without serum” and “RLU of positive control without serum minus RLU of negative control without virus and serum”. The formula was: (RLU _{control} - RLU ₛₑᵣᵤₘ)/(RLU ₚₒₛᵢₜᵢᵥₑ _{control} - RLU _{negative} _{control}). Fluorescence readings were measured by Tecan i-control (Infinite 500), and the RLU were calculated by GraphPad Prism v6.01. The 50% inhibition dilution titers (IC50) were the concentration of dilution that results in a >50% reduction in luciferase activity.

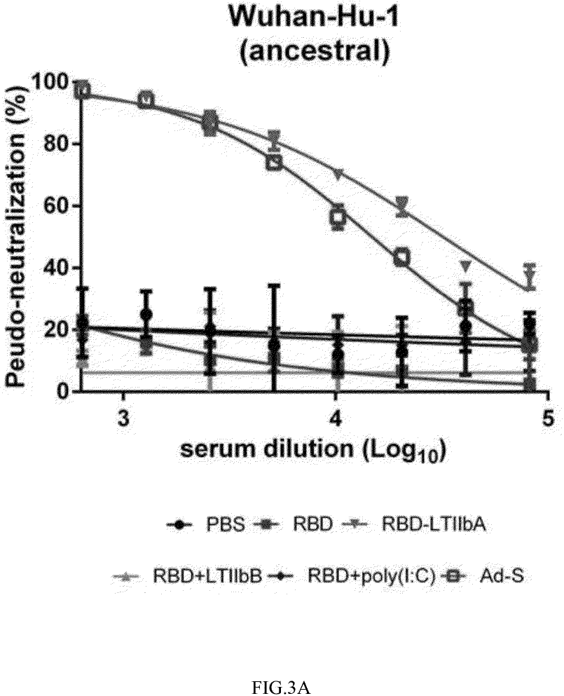

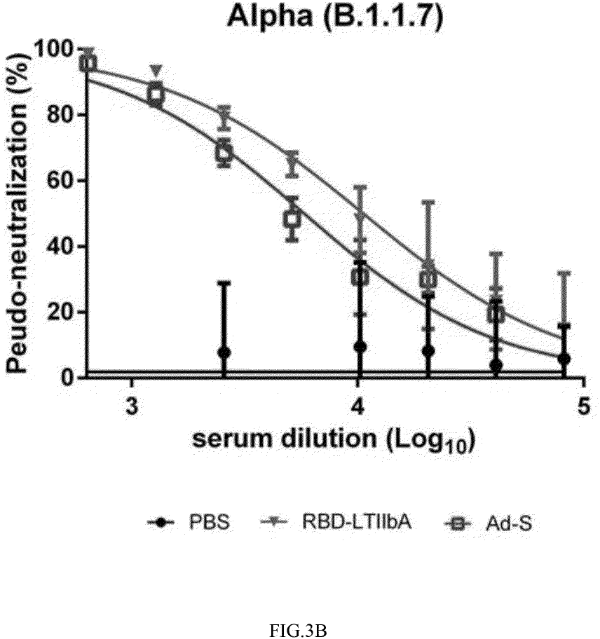

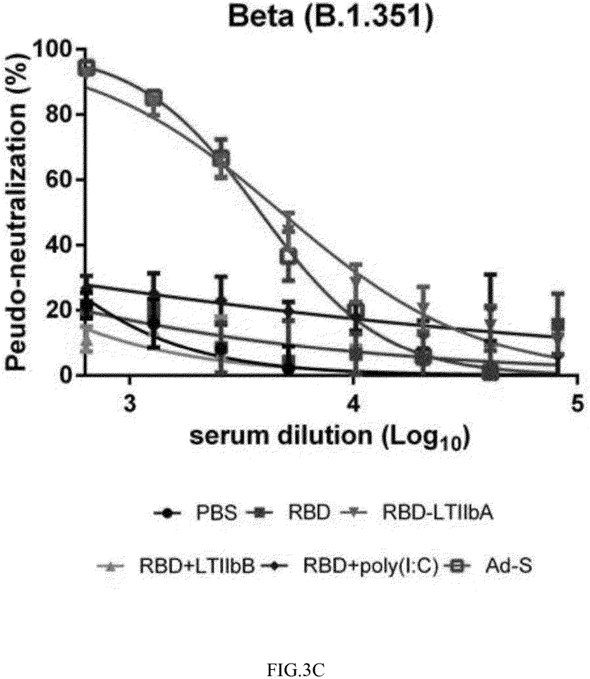

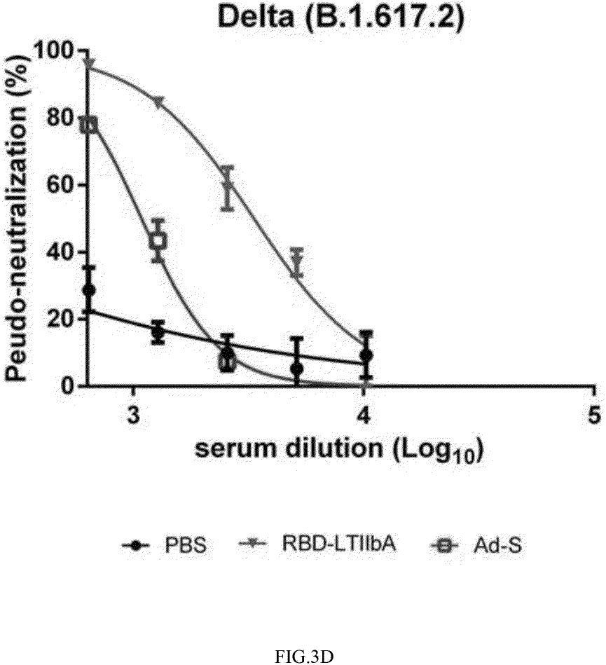

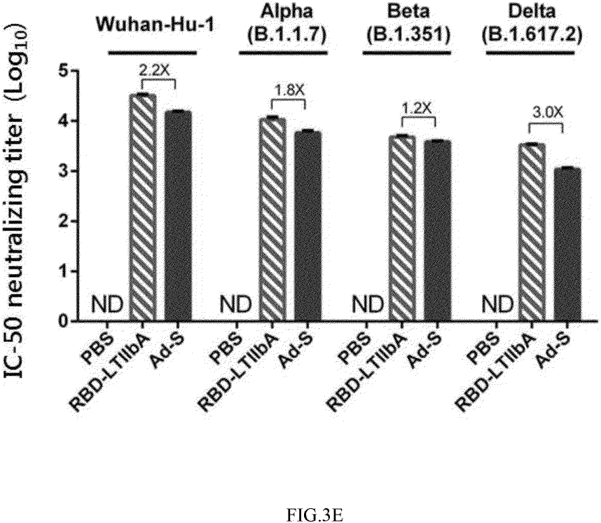

The neutralizing efficacy of the serum of mice after being intranasally immunized with the 3^{rd} dose of the mucosal vaccine composition containing the RBD-LTIIbA fusion protein of the present invention alone or the RBD protein together with different additional adjuvants against the original Wuhan-Hu-1 strain of SARS-CoV-2 of pseudovirus was shown as FIG. 3A, and the IC50 thereof was shown as FIG. 3E. Each data and the average thereof was shown in the FIGs. After mice were immunized with the mucosal vaccine composition containing the RBD-LTIIbA fusion protein of the present invention alone, the ability of the serum to neutralize the original Wuhan-Hu-1 strain of SARS-CoV-2 to inhibit cell infection was much higher than that of the serum from mice immunized with the mucosal vaccine composition containing the RBD protein alone or together with different adjuvants, and also higher than that of the serum from mice immunized with Ad-S of the positive control group, and IC50 thereof was 2.2 times higher than IC50 of the positive control group. The results indicated that being intranasal immunized with the mucosal vaccine composition of the present invention without additional adjuvants could produce a higher titer of neutralizing antibodies against the original Wuhan-Hu-1 strain of SARS-CoV-2, so as to effectively inhibit cells from infection of the original Wuhan-Hu-1 strain of SARS-CoV-2.

The neutralizing efficacy of the serum of mice after being intranasally immunized with the 3^{rd} dose of the mucosal vaccine composition containing the RBD-LTIIbA fusion protein of the present invention alone or the RBD protein together with different additional adjuvants against the Alpha mutant strain of SARS-CoV-2 (B.1.1.7) of pseudovirus was shown as FIG. 3B, and the IC50 thereof was shown as FIG. 3E. Each data and the average thereof was shown in the FIGs. After mice were immunized with the mucosal vaccine composition containing the RBD-LTIIbA fusion protein of the present invention alone, the ability of the serum to neutralize the Alpha mutant strain of SARS-CoV-2 (B.1.1.7) to inhibit cell infection was close to but slightly higher than that of the serum from mice immunized with Ad-S of the positive control group, and IC50 thereof was 1.8 times higher than IC50 of the positive control group. The results indicated that being intranasal immunized with the mucosal vaccine composition of the present invention without additional adjuvants could produce a higher titer of neutralizing antibodies against the Alpha mutant strain of SARS-CoV-2 (B.1.1.7), so as to effectively inhibit cells from infection thereof, and also indicated that the mucosal vaccine composition of the present invention provided the potential to fight against mutated SARS-CoV-2.

The neutralizing efficacy of the serum of mice after being intranasally immunized with the 3^{rd} dose of the mucosal vaccine composition containing the RBD-LTIIbA fusion protein of the present invention alone or the RBD protein together with different additional adjuvants against the Beta mutant strain of SARS-CoV-2 (B.1.351) pseudovirus was shown as FIG. 3C, and the IC50 thereof was shown as FIG. 3E. Each data and the average thereof were shown in the FIGs. After mice were immunized with the mucosal vaccine composition containing the RBD-LTIIbA fusion protein of the present invention alone, the ability of the serum to neutralize the Beta mutant strain of SARS-CoV-2 (B.1.351) to inhibit cell infection was higher than that of the serum from mice immunized with the mucosal vaccine composition containing the RBD protein alone or together with different adjuvants, and was close to but slightly higher than that of the serum from mice immunized with Ad-S of the positive control group, and IC50 thereof was 1.2 times higher than IC50 of the positive control group. The results indicated that being intranasal immunized with the mucosal vaccine composition of the present invention without additional adjuvants could produce a higher titer of neutralizing antibodies against the Beta mutant strain of SARS-CoV-2 (B.1.351), so as to effectively inhibit cells from infection thereof, and also indicated that the mucosal vaccine composition of the present invention provided the potential to fight against mutated SARS-CoV-2.

The neutralizing efficacy of the serum of mice after being intranasally immunized with the 3^{rd} dose of the mucosal vaccine composition containing the RBD-LTIIbA fusion protein of the present invention alone or the RBD protein together with different additional adjuvants against the Delta mutant strain of SARS-CoV-2 (B.1.617.2) of pseudovirus was shown as FIG. 3D, and the IC50 thereof was shown as FIG. 3E. Each data and the average thereof were shown in the FIGs. After mice were immunized with the mucosal vaccine composition containing the RBD-LTIIbA fusion protein of the present invention alone, the ability of the serum to neutralize the Delta mutant strain of SARS-CoV-2 (B.1.617.2) to inhibit cell infection was much higher than that of the serum from mice immunized with Ad-S of the positive control group, and IC50 thereof was 3.0 times higher than IC50 of the positive control group. The results indicated that being intranasal immunized with the mucosal vaccine composition of the present invention without additional adjuvants could produce a higher titer of neutralizing antibodies against the Delta mutant strain of SARS-CoV-2 (B.1.617.2), so as to effectively inhibit cells from infection thereof, and also indicated that the mucosal vaccine composition of the present invention provided the potential to fight against mutated SARS-CoV-2.

Together with all results of the embodiment, intranasal immunization with the mucosal vaccine composition of the present invention without additional adjuvants could significantly increase the amount of neutralizing antibodies against SARS-CoV-2 in mammals, and such excellent effects, which not only on the original Wuhan-Hu-1 strain of SARS-CoV-2 but also on the current three mutant strains of SARS-CoV-2, were better than that of the positive control group intranasal immunized with Ad-S. Therefore, the vaccine composition of the present invention containing the RBD-LTIIbA fusion protein provided great potential as a mucosal vaccine for mammals.

### Example 4

### The SARS-CoV-2 Mucosal Vaccine Composition Induces T-Cell Related Immune Response

Vaccines need to activate T helper cells to induce immunity of subjects, that is, to produce antibodies, and helper T cells contain T helper 1 cells (Th1 cells), T helper 2 cells (Th2 cells), and T helper 17 cells (Th17 cells); wherein, interferon-y (IFN-y) is an important cytokine for inducing Th1 immune responses; interleukin-5 (IL-5) is an important cytokine for inducing Th2 immune responses, and interleukin-17A (IL-17A) is an important cytokine for inducing Th17 immune responses and is an indicator of mucosal immune responses.

Therefore, in one embodiment of the present invention, in order to test the ability of the SARS-CoV-2 mucosal vaccine composition of the present invention to induce T-cell immune responses in subjects, T cells in the SPLs and the CLNs of mice immunized with the RBD-LTIIbA fusion protein of the present invention were collected; and after further stimulation with RBD proteins, the secretion of IFN-y, IL-5, and IL-17A from Th1 cells, Th2 cells, and Th17 cells in the culture medium of the T cells were detected.

The detailed experimental steps were as follows. First, in the manner described in Example 2, the following six groups were formulated into a 30 µL solution dosage form of vaccine composition: (1) the negative control group containing only PBS solution (represented as PBS); (2) the comparison group containing 20 µg of the RBD protein (represented as RBD); (3) the comparison group containing 20 µg of the RBD protein and 5 µg of LTIIb-B5 protein (represented as RBD+LTIIbB); (4) the experimental group containing 45 µg of the RBD-LTIIbA fusion protein of the present invention (the content of the RBD protein was 20 µg) (represented as RBD-LTIIbA); (5) the comparison group containing 20 µg of the RBD protein and 2 µg of poly(I:C) (represented as RBD+poly(I:C)); and (6) the positive control group containing 10⁸ PFU of the adenovirus vector, on which the spike protein of SARS-CoV-2 was expressed (represented as Ad-S). Next, the mice were immunized by nasal injection as described in Example 2. The mice were sacrificed at the 3^{rd} week after the last immunization, and the SPLs and the CLNs were collected and stimulated with RBD proteins as described above for subsequent experiments.

Next, SPLs and the CLNs that were collected from each group of mice and stimulated with RBD proteins were detected by ELISA to confirm the concentration of IFN-y, IL-5, and IL-17A. The following procedures for detecting the concentration of IFN-y, IL-5, and IL-17A were referred to the manufacturer’s instructions (Biolegend, IFN-y: 430801, IL-5: 431201, IL-17A: 432501). First, 100 µL of the diluted capture antibodies were coated onto 96-well culture plates in the coating buffer overnight at 4° C. The coating buffer was aspirated and each well was washed with 300 µL of PBST solution three times to wash off excess capture antibodies. Then, 250 µL of the assay diluent (1% BSA in PBS solution) was added into each well at room temperature for 2 hours to block non-specific binding. The assay diluent was aspirated and each well was washed with PBST solution four times to wash off excess assay diluent. Next, 100 µL of the commercial standard and the SPLs and the CLNs of each group of mice were properly diluted, and then the dilutions were added into the 96-well culture plates at room temperature for 2-hour shaking to allow IFN-y, IL-5, and IL-17A inside to bind to the capture antibodies. The dilutions were aspirated and each well was washed with PBST solution four times. Next, 100 µL of the detection antibodies were added into each well at room temperature for 1-hour shaking. The detection antibodies were aspirated and each well was washed with PBST solution four times to wash off excess detection antibodies. Then, 100 µL of diluted Avidin-HPR was added into each well at room temperature for 0.5-hour shaking. Each well was soaked and washed with PBST solution for 1 minute five times at intervals of 30 seconds. Finally, 100 µL of TMB was added into each well and incubated in the dark for 15 minutes. The reaction was stopped with 2 N H₂SO₄. The optical density at 450 nm was then measured by the ELISA analyzer (TECAN). Each concentration of the standard and the corresponding absorbance were used to draw a standard curve for calculating the concentration of IFN-y, IL-5, and IL-17A in SPLs and the CLNs of each group of mice.

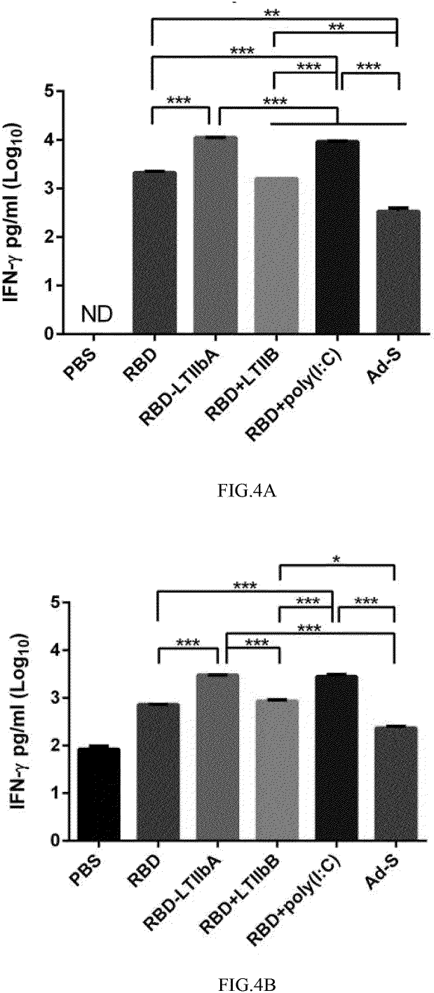

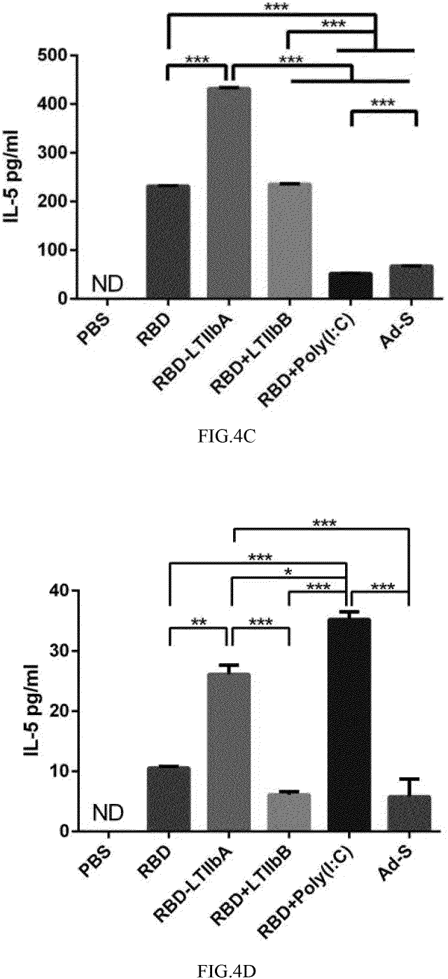

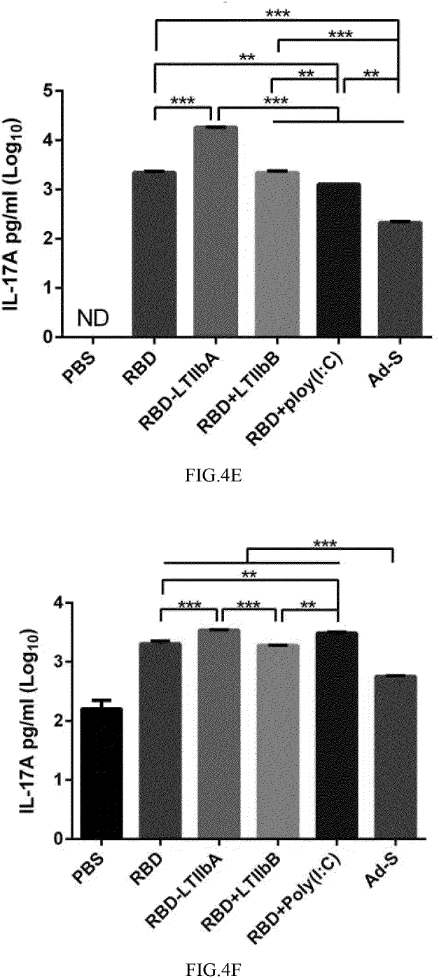

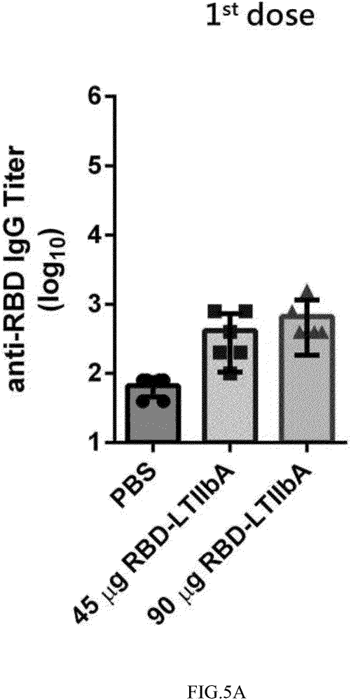

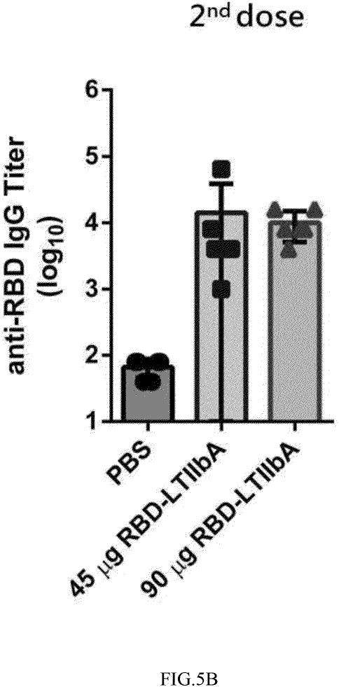

The concentration of IFN-y, IL-5, and IL-17A secreted by the SPLs, which were collected from mice intranasally immunized with the 3^{rd} dose of the mucosal vaccine composition containing the RBD-LTIIbA fusion protein of the present invention alone or the RBD protein together with different additional adjuvants, and further stimulated with RBD proteins of SARS-CoV-2, were shown as FIG. 4A, FIG. 4C, and FIG. 4E, respectively. Each data and the average thereof were shown in the FIGs. The concentration of IFN-y, IL-5, and IL-17A secreted by the CLNs thereof , which were further stimulated with RBD proteins of SARS-CoV-2, were shown as FIG. 4B, FIG. 4D, and FIG. 4F, respectively. Each data and the average thereof were shown in FIG. 4B, FIG. 4D, and FIG. 4F.

As shown in FIG. 4A, FIG. 4C, and FIG. 4E, after mice were immunized with the 3^{rd} dose of the mucosal vaccine composition containing the RBD-LTIIbA fusion protein of the present invention alone, the concentration of IFN-y, IL-5, and IL-17A secreted by the SPLs were all higher than these of the SPLs from mice immunized with the mucosal vaccine composition containing the RBD protein alone or together with different adjuvants, and even higher than these of the positive group, indicating that the SARS-CoV-2 mucosal vaccine composition of the present invention provided higher ability to induce T cells to produce cytokines. The results indicated that being intranasal immunized with the mucosal vaccine composition of the present invention without additional adjuvants could effectively induce T-cell related immune responses against SARS-CoV-2.

As shown in FIG. 4B, FIG. 4D, and FIG. 4F, after mice were immunized with the 3^{rd} dose of the mucosal vaccine composition containing the RBD-LTIIbA fusion protein of the present invention alone, the concentration of IFN-y, IL-5, and IL-17A secreted by the CLNs were all higher than these of the CLNs from mice immunized with the mucosal vaccine composition containing the RBD protein alone or together with LTIIbB5 adjuvant, and even higher than these of the positive group; and the concentration of IFN-y and IL-17A secreted by the CLNs were higher than these of the CLNs from mice immunized with the mucosal vaccine composition containing the RBD protein together with poly(I:C) adjuvant. The results indicated that the SARS-CoV-2 mucosal vaccine composition of the present invention could effectively induce T cells to produce cytokines, and being intranasal immunized with the mucosal vaccine composition of the present invention without additional adjuvants could effectively induce T-cell related immune responses against SARS-CoV-2.

### Example 5

### The SARS-CoV-2 Mucosal Vaccine Composition Induces Immune Responses in Hamsters

Since previous studies have pointed out that hamsters are highly susceptible to SARS-CoV-2 and are currently regarded as suitable preclinical animal models for developing vaccines, drugs, and other likes against SARS-CoV-2. Therefore, in one embodiment of the present invention, in order to test whether the SARS-CoV-2 mucosal vaccine composition of the present invention could effectively induce immune responses against SARS-CoV-2 in mammals without additional adjuvants, and to further observe the effects of dosage on induced immune responses, the vaccine composition containing the RBD-LTIIbA fusion protein of the present invention was injected into the nasal cavity of hamsters with three separated low or high doses, and two weeks after each immunization, the serum of hamsters was collected to test whether the serum contained antibodies against SARS-CoV-2.

First, PBS solution was used as a diluent to formulate the following three groups into a 100 µL solution dosage form of vaccine composition: (1) the negative control group containing only PBS solution (represented as PBS); (2) the low-dose experimental group containing 45 µg of the RBD-LTIIbA fusion protein of the present invention; (3) the high-dose experimental group containing 90 µg of the RBD-LTIIbA fusion protein of the present invention. Next, the three groups of vaccine composition were used to immunize female golden Syrian hamsters by nasal injection described above at the 0, 3^{rd}, and 6^{th} week, respectively, with at least six hamsters in each group. Serum samples of each group of hamsters were collected at the 2^{nd} week after each immunization to test whether there were antibodies against SARS-CoV-2 inside the serum of the hamsters.

Then, the serum samples collected from each group of hamsters were detected by ELISA to confirm there were IgG antibodies against SARS-CoV-2, so as to determine whether the immune responses of hamsters against SARS-CoV-2 were successfully elicited, and to further observe the effects of dosage and each immunization on the immune responses. First, 100 µL of the recombinant RBD protein with the concentration of 0.2 µg/well was coated onto a different 96-well ELISA plate (Thermo) in 0.05 M the coating buffer overnight at 4° C. The coating buffer was aspirated and each well was washed with 300 µL of PBST solution three times to wash off excess RBD proteins. The blocking buffer was added into each well at room temperature for 2 hours to block non-specific binding. The blocking buffer was aspirated and each well was washed with PBST solution three times to wash off excess blocking buffer. Then, the serum samples of each group of hamsters were serially 2-fold diluted with a dilution buffer solution starting from the original to 10000-time-diluted solution, and 100 µL of the serially diluted serum was added into the 96-well culture plate at room temperature for 1 hour to allow specific antibodies in the serum samples combined with the RBD protein. Each well was washed with PBST solution three times to wash off excess serum. Next, 100 µL of anti-hamsters IgG-HRP antibody (diluted in 1:30000) was added into each well of the 96-well culture plate at room temperature in the dark for 1 hours. Each well was washed three times with PBST solution to remove excess antibody. Finally, 100 µL of the substrate TMB of HRP was added to each well of the 96-well culture plate and incubated in the dark for 15 minutes, and then the reaction was stopped with 2 N H₂SO₄. The optical density at 450 nm was then measured by an ELISA analyzer (TECAN). The end-point titer of the anti-RBD IgG antibodies was calculated by a final serial dilution higher than 0.2 in the optical density value, and if the optical density value of the highest dilution exceeded 0.2 or the optical density value of the lowest dilution was less than 0.2, the experiment should be repeated at higher or lower dilution.

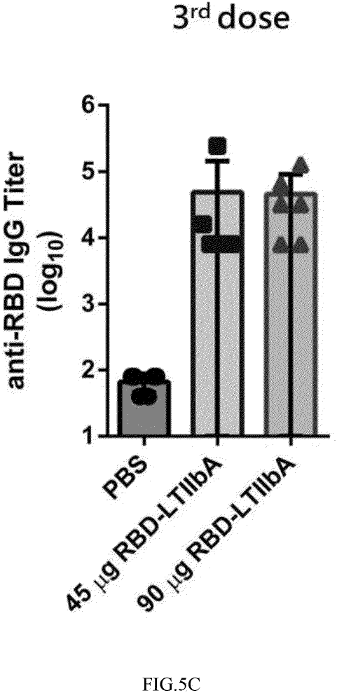

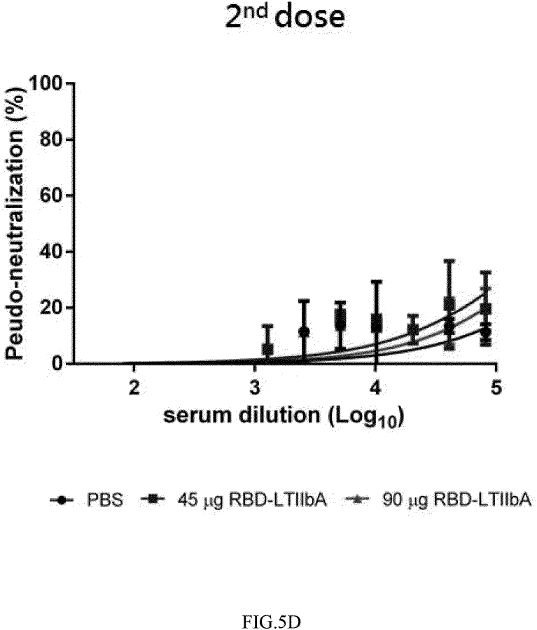

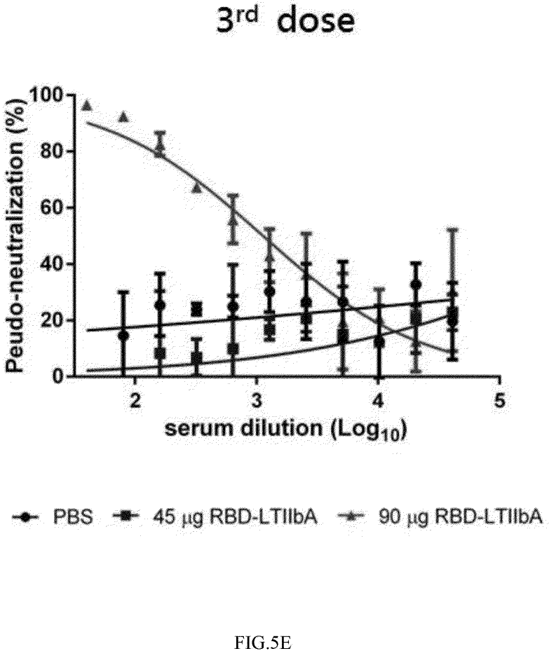

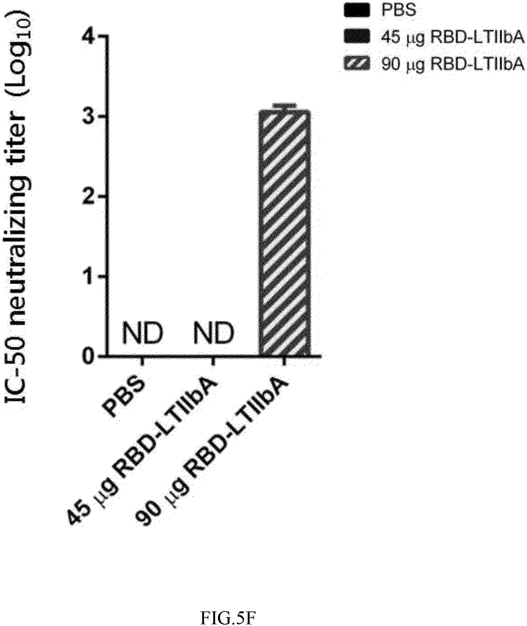

The titers of anti-RBD IgG antibodies in the serum of hamsters after being intranasally immunized with the 1^{st}, the 2^{nd}, and the 3^{rd} dose of the mucosal vaccine composition containing low-dose or high-dose of the RBD-LTIIbA fusion protein of the present invention were shown as FIG. 5A, FIG. 5B, and FIG. 5C, respectively. Each data and the average thereof were shown in the FIGs. * represented p <0.05; ** represented p <0.01; *** represented p <0.001; and **** represented p <0.0001. As shown in FIGS. 5A to 5C, no matter being immunized with the 1^{st} dose of the mucosal vaccine composition containing low-dose or high-dose RBD-LTIIbA fusion protein of the present invention, the hamsters could produce IgG antibody with greater than 10² titers; and after being immunized with the 2^{nd} dose, the hamsters could produce IgG antibody with about 10⁴ titers; and after being immunized with the 3^{rd} dose, the hamsters could produce IgG antibody with greater than 10⁴ titers. The results indicated that being intranasal immunized with the mucosal vaccine composition of the present invention without additional adjuvants could effectively induce hamsters to produce antibodies against SARS-CoV-2.

In the embodiment of the present invention, in order to further observe effects of dosage on neutralizing antibody titer induced by the SARS-CoV-2 mucosal vaccine composition of the present invention, the pseudovirus neutralization assay was used to test the efficacy of neutralizing antibodies produced by hamsters after being immunized with each of the aforementioned three separated low or high doses of the SARS-CoV-2 mucosal vaccine composition of the present invention on neutralizing the infection of SARS-CoV-2.

Before testing the ability of the serum samples to neutralize infection of viruses, 2×10⁴ HEK-293T cells stably expressing hACE2 were seeded in each well of a 96-well culture plate at 37° C. for one day; wherein, DMEM containing 1% FBS was used as the cell culture medium. The aforementioned serum samples were serially 2-fold diluted with MEM containing 2% FBS from the 30-fold-diluted solution. Each diluted serum was respectively incubated with 1,000 TU of the original Wuhan-Hu-1 strain of SARS-CoV-2 pseudovirus for 1 hour at 37° C. The mixture of the serum and the pseudovirus was then added into the aforementioned 96-well culture plate to infect the cells; wherein, the volume of the added mixture was equal to the volume of the culture medium in the culture plate, so the dilution fold would increase (starting at 40-fold). After the HEK-293T cells expressing hACE2 were infected at 37° C. for 1 hour, the culture medium was replaced with fresh DMEM containing 10% FBS. The HEK-293T cells were continuously cultured in the 37° C. incubator until 72 hours post-infection. The fresh cell culture medium should be replaced daily. The cells were lysed and subjected to luciferase assay (Promega Bright-GloTM Luciferase Assay System) to calculate the ability of serum from each group of hamsters to neutralize the viruses. The inhibition percentage of viral infection was calculated by “losing relative luciferase units (RLU) between serum and positive control without serum” and “RLU of positive control without serum minus RLU of negative control without virus and serum”. The formula was: (RLU_{control} - RLU ₛₑᵣᵤₘ)/(RLU ₚₒₛᵢₜᵢᵥₑ _{control} - RLU _{negative} _{control}). Fluorescence readings were measured by Tecan i-control (Infinite 500), and the RLU were calculated by GraphPad Prism v6.01. The 50% inhibition dilution titers (IC50) were the concentration of dilution that results in a >50% reduction in luciferase activity.

The neutralizing efficacy of the serum of hamsters after being intranasally immunized with the 2^{nd} dose of mucosal vaccine composition containing the RBD-LTIIbA fusion protein of the present invention against SARS-CoV-2 of pseudovirus was shown as FIG. 5D; the result of the serum of hamsters after being intranasally immunized with the 3^{rd} dose was shown as FIG. 5E; and the IC50 thereof was shown as FIG. 5F. Each data and the average thereof were shown in the FIGs. As shown in FIGS. 5D to 5F, after being immunized with the 3^{rd} high-dose of the mucosal vaccine composition of the present invention, the hamsters could effectively produce neutralizing antibody against SARS-CoV-2, and the titer was about 10³. The results indicated that being intranasal immunized with the mucosal vaccine composition of the present invention without additional adjuvants could produce a higher titer of neutralizing antibodies against SARS-CoV-2, so as to effectively inhibit cells from infection of SARS-CoV-2.

In one embodiment of the present invention, in order to further observe the efficacy of the SARS-CoV-2 mucosal vaccine composition of the present invention on inducing immune responses for directly inhibiting the infection of SARS-CoV-2, the virus challenge trials were used to test the ability of hamsters to resist infection of SARS-CoV-2 after being immunized with the three separated low or high doses of the SARS-CoV-2 mucosal vaccine composition of the present invention.

First, at the 7^{th} week after being immunized with the 3^{rd} dose of the vaccine composition containing the RBD-LTIIbA fusion protein of the present invention (i.e. the 13th week after the start of the experiment), hamsters were challenged with 10⁴ PFU of SARS-CoV-2 (hCoV-19/Taiwan/4/2020). For each hamster, the virus was conducted intranasally in a volume of 100 µL. The body weight and survival rates were tracked through the next 6 days. The hamsters were sacrificed at the 3^{rd} and 6^{th} days post-infection (dpi) for lung tissue and nasal irrigation sampling. The fifty-percent tissue culture infectious dose (TCID50) was used for virus titration, and the lung tissue was stained to observe the morphology thereof.

In the virus titration experiment, the lung tissues collected from hamsters were homogenized in 600 µL of DMEM (2% FBS, 1% penicillin/streptomycin). The supernatant was separated from the homogenate by centrifugation at 15,000 rpm for 5 minutes. The supernatant was taken for the virus titration. The method of titrating virus with TCID50 was briefly described as follows. Confluent monolayers of Vero E6 cells were infected by serial dilutions of each sample and incubated for another 4 days. Then, the Vero E6 cells were fixed with 10% formaldehyde and stained with 0.5% crystal violet for 20 minutes. The excessive crystal violet was washed out with water and evaluated for TCID50/mL using Reed and Muench method.

The lung tissues collected from hamsters were fixed with 10% neutral buffered formalin for 24 hours. The fixed tissues were embedded with paraffin and trimmed to 5 mm in thickness. The samples were stained with hematoxylin and eosin (H & E). The stained lung tissues of each group of hamsters were observed and recorded with an optical microscope.

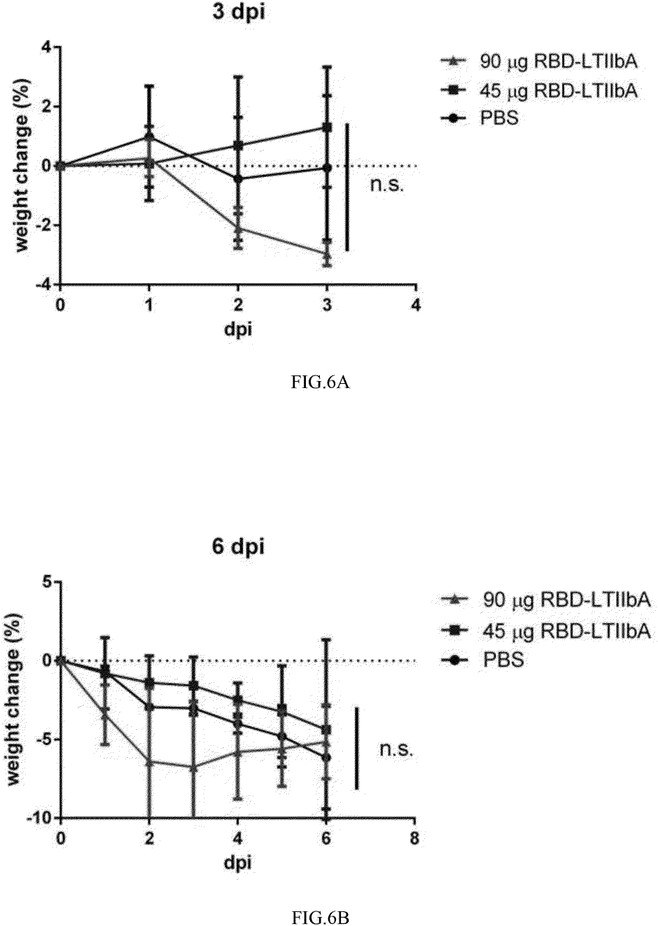

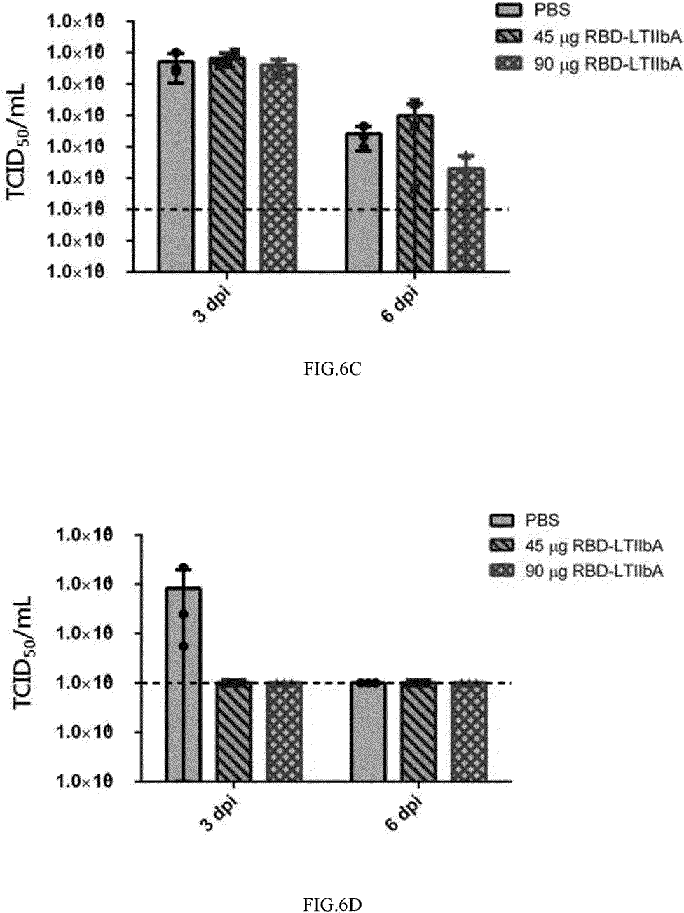

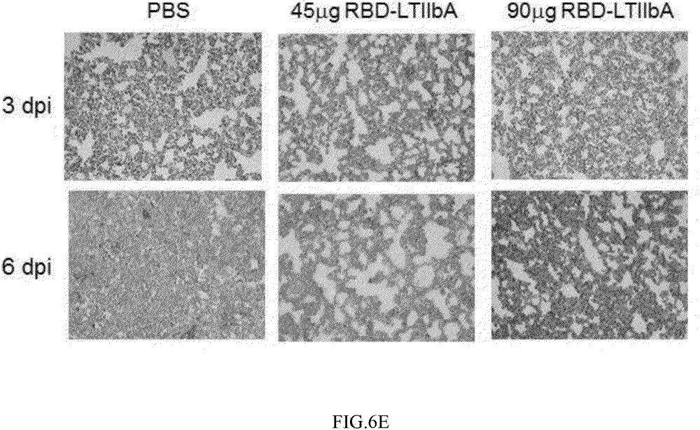

After being immunized with three doses of the mucosal vaccine composition containing the RBD-LTIIbA fusion protein of the present invention, the body weight change of hamsters that were further challenged with SARS-CoV-2 for three days was shown as FIG. 6A, and the body weight change of hamsters that were further challenged with SARS-CoV-2 for six days was shown as FIG. 6B. As shown in FIGS. 6A and 6B, there was no significant difference between the body weight of hamsters immunized with the mucosal vaccine composition of the present invention and the body weight of hamsters without immunization.

After being immunized with three doses of the mucosal vaccine composition containing the RBD-LTIIbA fusion protein of the present invention, the TCID50 of the lung tissues of hamsters which were further challenged with SARS-CoV-2 was shown as FIG. 6C, and TCID50 of the nasal irrigation of the hamsters was shown as FIG. 6D, and the stained lung tissues of the hamsters was shown as FIG. 6E. As shown in FIGS. 6C and 6D, in the lung tissues, hamsters immunized with 90 µg of the RBD-LTIIbA fusion protein of the present invention had the lowest viral titration on the 6^{th} day after the SARS-CoV-2 challenge; and in the nasal irrigation, no matter hamsters immunized with 45 µg or 90 µg of the RBD-LTIIbA fusion protein of the present invention showed low viral titration on the 3^{rd} days after the SARS-CoV-2 challenge. The results indicated that immunized with the RBD-LTIIbA fusion protein of the present invention could produce protective effects on the mucosa of subjects to effectively resist the infection of SARS-CoV-2.

As shown in FIG. 6E, without immunized with the RBD-LTIIbA fusion protein of the present invention, the lung tissues of hamsters showed cell infiltration on the 6^{th} day after the SARS-CoV-2 challenge, and no matter immunized with 45 µg or 90 µg of the RBD-LTIIbA fusion protein of the present invention, the cell infiltration of the lung tissues of hamsters could be significantly reduced. The results indicated that immunized with the RBD-LTIIbA fusion protein of the present invention could produce protective effects on the lung tissues of subjects to effectively resist the infection of SARS-CoV-2.

In summary, in the SARS-CoV-2 mucosal vaccine composition of the present invention, a protein subunit of the RBD-LTIIbA fusion protein is used as an antigen to form a vaccine composition with a built-in adjuvant. In the case of fusion with LTIIbA, the structure of the RBD-LTIIbA fusion protein of the present invention will not affect the binding stability between the RBD and ACE2. When the SARS-CoV-2 mucosal vaccine composition of the present invention is applied to a subject, such as nasal injection, the SARS-CoV-2 mucosal vaccine composition can effectively elicit the humoral and cellular immune responses against SARS-CoV-2 in the subject without any additional adjuvants, including eliciting antigen-specific and the neutralizing IgG antibodies and IgA antibodies in blood and broncho-alveolar mucosa against SARS-CoV-2, and increasing cytokines secreted by T cells such as IFN-y, IL-5, and IL-17A, to effectively enhance the ability of the subject to prevent the attack/infection of SARS-CoV-2. The SARS-CoV-2 mucosal vaccine composition of the present invention can effectively elicit systemic immunity and mucosal immunity as well, and especially stimulate mucosal immune responses of the respiratory tract, so as to provide the first-line protection against foreign pathogens directly. Therefore, the SARS-CoV-2 mucosal vaccine composition of the present invention can provide subjects with effective protection against SARS-CoV-2 infection.

What is claimed is:

1. A severe acute respiratory syndrome coronavirus-2 (SARS-CoV-2) mucosal vaccine composition, comprising an antigen fusion protein, wherein the antigen fusion protein comprises a SARS-CoV-2 antigen and a Type IIb heat-labile enterotoxin A subunit from an Escherichia coli.

2. The SARS-CoV-2 mucosal vaccine composition according to claim 1, wherein the SARS-CoV-2 antigen is a spike protein.

3. The SARS-CoV-2 mucosal vaccine composition according to claim 2, wherein the SARS-CoV-2 antigen is a receptor-binding domain (RBD) of the spike protein.

4. The SARS-CoV-2 mucosal vaccine composition according to claim 3, wherein a binding stability between the RBD and an angiotensin-converting enzyme 2 (ACE2) is not affected by a structure of the antigen fusion protein.

5. The SARS-CoV-2 mucosal vaccine composition according to claim 3, wherein an N-terminal region of the SARS-CoV-2 antigen further comprises a poly-histidine segment and/or a signal peptide segment of an envelope glycoprotein.

6. The SARS-CoV-2 mucosal vaccine composition according to claim 5, wherein a binding stability between the RBD and an ACE2 is not affected by a structure of the antigen fusion protein.

7. The SARS-CoV-2 mucosal vaccine composition according to claim 1, wherein the SARS-CoV-2 mucosal vaccine composition comprises at least 45 µg of the antigen fusion protein.

8. A method of treating or preventing SARS-CoV-2 infection, comprising administering to a subject in need thereof a SARS-CoV-2 mucosal vaccine composition comprising an effective amount of the antigen fusion protein according to claim 1.

9. The method according to claim 8, wherein the antigen fusion protein elicits high titer antigen-specific antibodies and/or neutralizing antibodies against SARS-CoV-2.

10. The method according to claim 9, wherein the antigen-specific antibodies and the neutralizing antibodies are IgG antibodies and/or IgA antibodies.

11. The method according to claim 8, wherein the antigen fusion protein elicits a T-cell related immune response.

12. The method according to claim 11, wherein the T-cell related immune response includes secretions of interferon-γ (IFN-γ), interleukin-5 (IL-5), interleukin-17A (IL-17A), or any combinations thereof.

13. The method according to claim 8, wherein the SARS-CoV-2 mucosal vaccine composition is administered through a nasal cavity of the subject in need thereof.

14. A method of preparing a SARS-CoV-2 mucosal vaccine composition, comprising the steps of:
(a) preparing the antigen fusion protein according to claim 1; and
(b) mixing the antigen fusion protein with a pharmaceutically acceptable carrier to obtain a SARS-CoV-2 mucosal vaccine composition.

15. The method according to claim 14, wherein the SARS-CoV-2 mucosal vaccine composition comprises at least 45 µg of the antigen fusion protein.
